(19) United States
(12) Patent Application Publication
(10) Pub. No.: US 2025/0325495 A1
(43) Pub. Date: Oct. 23, 2025

(54) SILK NANOPARTICLE SYNTHESIS: TUNING SIZE, DISPERSITY, AND SURFACE CHEMISTRY FOR DRUG DELIVERY

(71) Applicant: Trustees of Tufts College, MEDFORD, MA (US)

(72) Inventors: Sawnaz Shaidani, Pelham, NH (US); Thomas Falcucci, Boston, MA (US); Olivia Foster, Medford, MA (US); Jugal Kishore Sahoo, Belmont, MA (US); Onur Hasturk, Medford, MA (US); David L. Kaplan, Concord, MA (US); Charlotte S. Jacobus, Brooklyn, NY (US)

(21) Appl. No.: 19/198,560

(22) Filed: May 5, 2025

Related U.S. Application Data
(63) Continuation of application No. PCT/US23/78662, filed on Nov. 3, 2023.
(60) Provisional application No. 63/382,485, filed on Nov. 4, 2022.
(60) Provisional application No. 63/382,716, filed on Nov. 7, 2022.

(51) Int. Cl. A61K 9/51; A61K 31/704

(52) U.S. Cl. CPC A61K 9/5192; A61K 9/5169; A61K 31/704

(57) ABSTRACT

Methods disclosed herein relate to generating silk nanoparticles with a low polydispersity index (PDI). Methods include adding a silk solution dropwise into a volatile solvent that is miscible with water, thereby forming a precipitate-bearing solution and applying shear forces to the precipitate-bearing solution for a length of time sufficient to achieve evaporation of at least 95% of the volatile solvent, thereby producing a population of silk fibroin nanoparticles in water having a polydispersity index (PDI) of 0.35 or less, including but not limited to, a PDI of 0.330 or less, 0.325 or less, 0.315 or less, 0.310 or less, 0.30 or less, 0.275 or less, 0.250 or less, 0.225 or less, or 0.200 or less. Parameters related to silk fibroin molecular weight, silk fibroin concentration, shear force application, and temperature may all be modified to achieve silk nanoparticles of a particular size exhibiting particular PDIs.

### CLAIM TO PRIORITY

This application claims benefit of and is a continuation of International Patent Application No. PCT/US2023/078662 (Attorney Docket. No. 2095.0573), filed Nov. 3, 2023, and entitled “SILK NANOPARTICLE SYNTHESIS: TUNING SIZE, DISPERSITY, AND SURFACE CHEMISTRY FOR DRUG DELIVERY,” International Pub. No. WO 2024097977, which is hereby incorporated by reference in its entirety for all purposes.

International Patent Application No. PCT/US2023/078662 claims the benefit of the following provisional applications which are hereby incorporated by reference in their entirety for all purposes: U.S. Patent Application Ser. No. 63/382,485 (Attorney Docket. No. 2095.0421), filed Nov. 4, 2022; and U.S. Patent Application Ser. No. 63/382,716 (Attorney Docket. No. 2095.0422), filed Nov. 7, 2022.

### GOVERNMENT FUNDING STATEMENT

This invention was made with government support under FA9550-20-1-0363 awarded by the United States Air Force and P41EB027062 awarded by the National Institutes of Health. The government has certain rights in the invention.

### BACKGROUND

Protein-based nanoparticles as carriers for drug-delivery are of interest to traverse different biological (e.g., systemic or microenvironmental, etc.) barriers and enable targeted delivery. Silk protein-based nanoparticles are useful and versatile drug delivery systems for sustained and controlled release due to their biocompatibility, biodegradability, accessible chemistries, and ability to stabilize different drugs and other biomolecules.

### SUMMARY

In the present study, silk nanoparticles (SNPs) were engineered using a nanoprecipitation technique with tight control over size (˜45-250 nm diameter) with low polydispersity by altering variables including stirring speed, reaction bath temperature, silk molecular weight (MW), and silk concentration. Of these variables, stir speed was a significant contributor towards particle size control. SNPs with positive or negative surface charges and decoration with surface antigens were also demonstrated. New mechanistic insight into control of SNP size and corresponding polydispersity index (PDI), cellular uptake using glioblastoma as a model, surface characteristics (e.g., mechanical properties when added to varying matrices (gels, microneedles), and the entrapment of small molecule drugs (e.g., doxorubicin, small hydrophobic drugs, etc.) within the particles are disclosed. These insights expand the potential utility of SNPs for medical/drug delivery, environmental (e.g., plant uptake, pesticide distribution, etc.), matrix designs, consumer products (e.g., oils, colorants, fragrances, etc.), and food applications (e.g., flavor, vitamin, and taste ingredient storage and release; shelf stability, etc.).

In some aspects, the techniques described herein relate to a method including: a) adding a silk solution dropwise into a volatile solvent that is miscible with water, thereby forming a precipitate-bearing solution, wherein the silk solution contains silk fibroin in an amount by weight of at least 2%, at least 3%, at least 5%, at least 6%, or at least 7%, and at most 25%, wherein the precipitate-bearing solution includes the organic solvent in an amount of at least 75% (v/v); b) applying shear forces to the precipitate-bearing solution, and the stirring continuing for a length of time sufficient to achieve evaporation of at least 95% of the volatile solvent, thereby producing a population of silk fibroin nanoparticles in water having a polydispersity index (PDI) of 0.35 or less, including but not limited to, a PDI of 0.330 or less, 0.325 or less, 0.315 or less, 0.310 or less, 0.30 or less, 0.275 or less, 0.250 or less, 0.225 or less, or 0.200 or less.

In some aspects, the techniques described herein relate to a method of administering silk fibroin nanoparticles including administering a first plurality of silk fibroin nanoparticles having an average diameter below a predetermined size threshold and a polydispersity index of 0.35 or less, including but not limited to, a PDI of 0.330 or less, 0.325 or less, 0.315 or less, 0.310 or less, 0.30 or less, 0.275 or less, 0.250 or less, 0.225 or less, or 0.200 or less, wherein the predetermined size threshold determines intracellular mobility.

In some aspects, the techniques described herein relate to a method of tuning silk nanoparticle size, including: obtaining a silk solution, the silk solution including silk fibroin of a selected molecular weight and a selected concentration; adding the silk solution dropwise into a volatile solvent that is miscible with water, thereby forming a precipitate-bearing solution, wherein the precipitate-bearing solution includes the organic solvent in an amount of at least 75% (v/v); applying shear forces to the precipitate-bearing solution, the applying continuing for a length of time sufficient to achieve evaporation of at least 95% of the volatile solvent, thereby producing a population of silk fibroin nanoparticles in water having a polydispersity index (PDI) of 0.35 or less, including but not limited to, a PDI of 0.330 or less, 0.325 or less, 0.315 or less, 0.310 or less, 0.30 or less, 0.275 or less, 0.250 or less, 0.225 or less, or 0.200 or less.

In some aspects, the techniques described herein relate to compositions made by any of the methods disclosed herein.

These and other systems, methods, objects, features, and advantages of the present disclosure will be apparent to those skilled in the art from the following detailed description of the preferred embodiment and the drawings.

All documents mentioned herein are hereby incorporated in their entirety by reference. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context.

### BRIEF DESCRIPTION OF THE FIGURES

The disclosure and the following detailed description of certain embodiments thereof may be understood by reference to the following figures:

FIG. 1 depicts a schematic of applications of nanoprecipitated silk particles is shown. Silk fibroin is extracted from Bombyx mori cocoons, and following particle fabrication (e.g., nanoprecipitation with organic solvent with or without drug, using native or positively charged silk), particles can be altered to display surface targeting agents (antibodies) and incubated with cells for cellular uptake studies. SNPs generated are: silk nanoparticles, ESNPs: particles with entrapped drug, CSNPs: particles coated with drug.

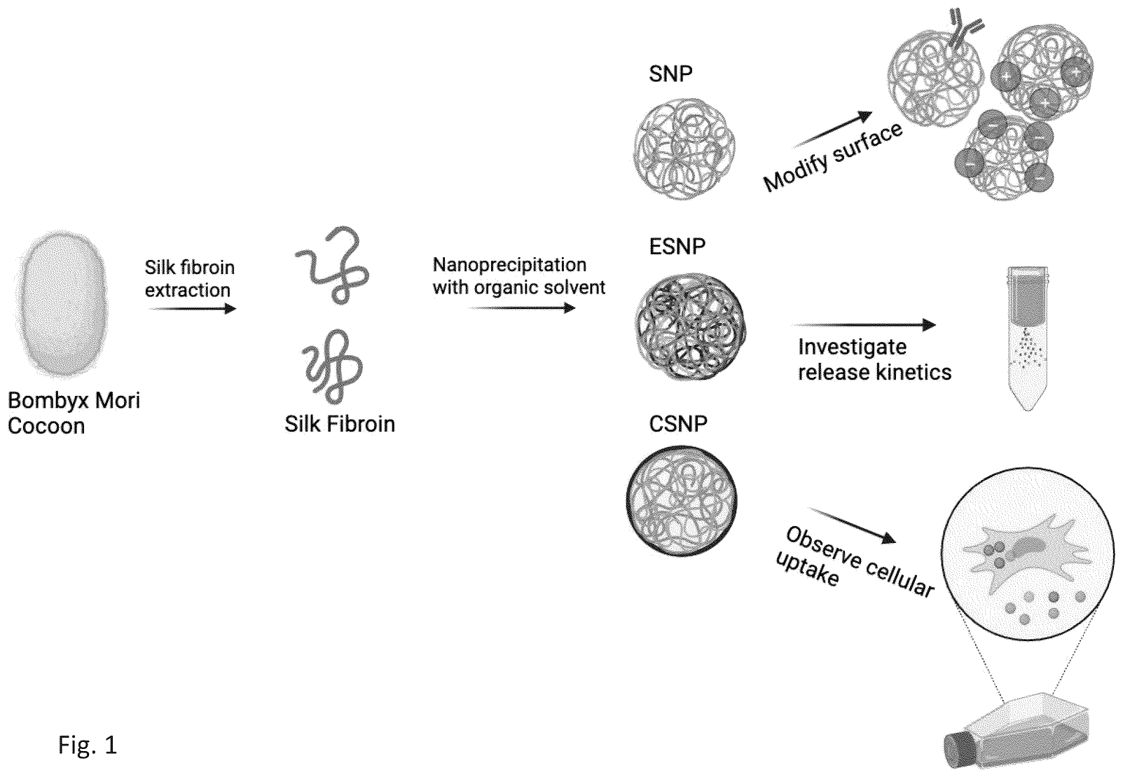

FIG. 2A depicts the average diameter of SNPs of different sizes with size distribution as determined by Dynamic Light Scattering (DLS) generated by an example nanoprecipitation technique. Panel i of FIG. 2A is an SEM of 130 nm particles formed at 200 rpm, 6% silk, mid MW. Panel ii of FIG. 2A are TEM images of 90 nm particles, formed using 1,000 rpm stir speed, 7% silk, and mid MW. Panel iii of FIG. 2A are TEM images of 65 nm particles, formed using 1,200 rpm stir speed, 5% silk, and mid MW. Sizes of technical replicates were averaged and then graphed for each biological replicate (see methods, n≥3 biological replicates). FIG. 2B depicts an example nanoprecipitation protocol, where silk solution is added dropwise to an acetone bath, with specific silk concentration, MW, and stir rate, followed by removal acetone (e.g., such as by centrifugation/evaporation), and then characterization (e.g., such as by DLS/SEM to confirm size). FIG. 2C is a plot of the polydispersity index (PDI) vs. sample prep conditions for SNPs of various sizes generated using different formulation combinations (as seen in FIG. 2A). PDIs of technical replicates were averaged and then graphed for each biological replicate (see methods, n≥3 biological replicates). FIG. 2D depicts the following: FIG. 2Di. Particles formed using the same concentrations and MWs, but different stir speeds; FIG. 2Dii. Particles formed using different silk MWs; FIG. 2Diii. Particles formed using the same stir speed and MW, but different concentrations; and FIG. 2Div. Nanoprecipitation done at colder temperatures to induce size differences, while maintaining the molecular weight, concentration, and stir speeds. FIG. 2E depicts principal component analysis (PCA) of the variables PC1=stir speed, PC2, =Concentration, PC3=Molecular weight. 60-minute extraction: low MW (<171 kDa), 30 min extraction: mid MW (31-268 kDa), 10 min extraction: high MW (171-460 kDa). Error bars represent standard deviation, n≥3.

FIG. 3A depicts size differences in blank, unloaded particles vs. particles with pre-dissolved doxorubicin prior to nanoprecipitation (entrapment, ESNP). FIG. 3B depicts loading of doxorubicin in entrapped drug SNPS (ESNPs) vs. particles coated with doxorubicin (CSNPs) per mg of SNPs. Fig. C depicts percent doxorubicin released in entrapped drug SNPS (ESNPs) vs. particles coated with doxorubicin (CSNPs) over time. Error bars represent standard deviation, n=3. FIG. 3D depicts an in vitro release profile of doxorubicin from ESNPs of different sizes compared to CSNPs. Error bars represent standard deviation, n=3.

FIGS. 4A-4C depict: live cell imaging of fluorescently (FITC, green) tagged nanoparticles 130 nm (FIG. 4A), 78 nm (FIG. 4B), or 65 nm (FIG. 4C) in U87-MG glioblastoma cells with stained lysosomes (Lysotracker, red). Overlayed images show colocalization of particles with the lysosomes in orange/yellow. Scale bars=200 um, images taken in 10× with same exposure levels, n≥3. FIGS. 4D & 4E depict confocal, fixed imaging of fluorescently (FITC, green) tagged nanoparticles that are 130 nm (FIG. 4D) or 65 nm (FIG. 4E) in U87-MG glioblastoma cells with stained lysosomes (Lamp2, blue), early endosomes (EEA1, blue) and phalloidin (red). Colocalization of the particles with endosomes/lysosomes cyan colored, colocalization of SNPs with the cytoskeleton (phalloidin) yellow. Arrows indicate potential areas where particles do not colocalize with any labeled regions. Scale bars=20 uM, images are 40×. n≥3.

FIG. 5 depicts enhancement materials properties of SNPs. FIG. 5A) Altered zeta potential after amination of silk solution prior to nanoprecipitation to generate a positive surface charge in comparison to native (unmodified) silk nanoparticles (n=3). FIG. 5B) Relative fluorescent units (RFU) of fluorescent secondary antibody attached to primary antibody conjugated to the surface of the SNPs, compared to non-specific binding in the “Only Secondary Control” (n=2 for this antibody, all technical replicates plotted). All values were normalized against blank SNPs. C) i. Fluorescent NP aggregates after secondary antibody incubation with conjugated NPs compared to ii. Nonspecific binding of blank particles incubated with secondary antibody, without primary antibody conjugated to the surface (n=2). Error bars represent standard deviation. Scale bars=300 um. Exposure levels used to take images were the same in all images.

FIG. 6 depicts the modulus of silk fibroin hydrogels with or without nanoparticles embedded in the gels, analyzed with dynamic mechanical analysis. Error bars represent standard deviation. (n≥3).

FIG. 7 depicts particles formed all at 1200 rpm stir speed, altering magnet size and molecular weight. A larger magnet (12×9 mm) induced greater shear stress, producing smaller particles.

### DETAILED DESCRIPTION

Before the present disclosure is described in further detail, it is to be understood that the disclosure is not limited to the particular embodiments described. It is also understood that the terminology used herein is for the purpose of describing particular embodiments only, and is not intended to be limiting. The scope of the present disclosure will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

Substantially: as used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Silk is a natural protein fiber produced in a specialized gland of certain organisms. Silk production in organisms is especially common in the Hymenoptera (bees, wasps, and ants), and is sometimes used in nest construction. Other types of arthropod also produce silk, most notably various arachnids such as spiders (e.g., spider silk). Silk fibers generated by insects and spiders represent the strongest natural fibers known and rival even synthetic high performance fibers.

Silk has been a highly desired and widely used textile since its first appearance in ancient China (see Elisseeff, “The Silk Roads: Highways of Culture and Commerce,” Berghahn Books/UNESCO, New York (2000); see also Vainker, “Chinese Silk: A Cultural History,” Rutgers University Press, Piscataway, New Jersey (2004)). Glossy and smooth, silk is favored by not only fashion designers but also tissue engineers because it is mechanically tough but degrades harmlessly inside the body, offering new opportunities as a highly robust and biocompatible material substrate (see Altman et ah, Biomaterials, 24: 401 (2003); see also Sashina et ah, Russ. J. Appl. Chem., 79: 869 (2006)).

Silk is naturally produced by various species, including, without limitation: Antheraea mylitta; Antheraea pernyi; Antheraea yamamai; Galleria mellonella; Bombyx mori; Bombyx mandarina; Galleria mellonella; Nephila clavipes; Nephila senegalensis; Gasteracantha mammosa; Argiope aurantia; Araneus diadematus; Latrodectus geometricus; Araneus bicentenarius; Tetragnatha versicolor; Araneus ventricosus; Dolomedes tenebrosus; Euagrus chisoseus; Plectreurys tristis; Argiope trifasciata; and Nephila madagascariensis.

As is known in the art, silks are modular in design, with large internal repeats flanked by shorter (−100 amino acid) terminal domains (N and C termini). Naturally-occurring silks have high molecular weight (200 to 350 kDa or higher) with transcripts of 10,000 base pairs and higher and >3000 amino acids (reviewed in Omenatto and Kaplan (2010) Science 329: 528-531). The larger modular domains are interrupted with relatively short spacers with hydrophobic charge groups in the case of silkworm silk. N- and C-termini are involved in the assembly and processing of silks, including pH control of assembly. The N- and C-termini are highly conserved, in spite of their relatively small size compared with the internal modules.

As used herein, “silk fibroin” refers to silk fibroin protein whether produced by silkworm, spider, or other insect, or otherwise generated (Lucas et al., Adv. Protein Chem., 13:107-242 (1958)). Any type of silk fibroin can be used in different embodiments described herein. Silk fibroin produced by silkworms, such as Bombyx mori, is the most common and represents an earth-friendly, renewable resource. For instance, silk fibroin used in a silk film may be attained by extracting sericin from the cocoons of B. mori. Organic silkworm cocoons are also commercially available. There are many different silks, however, including spider silk (e.g., obtained from Nephila clavipes), transgenic silks, genetically engineered silks, such as silks from bacteria, yeast, mammalian cells, transgenic animals, or transgenic plants, and variants thereof, that can be used. See, e.g., WO 97/08315 and U.S. Pat. No. 5,245,012, each of which is incorporated herein by reference in their entireties.

In general, silk fibroin for use in accordance with the present invention may be produced by any such organism, or may be prepared through an artificial process, for example, involving genetic engineering of cells or organisms to produce a silk protein and/or chemical synthesis. In some embodiments of the present invention, silk fibroin is produced by the silkworm, Bombyx mori. Fibroin is a type of structural protein produced by certain spider and insect species that produce silk. Cocoon silk produced by the silkworm, Bombyx mori, is of particular interest because it offers low-cost, bulk-scale production suitable for a number of commercial applications, such as textile.

Silkworm cocoon silk contains two structural proteins, the fibroin heavy chain (−350 kDa) and the fibroin light chain (˜25 kDa), which are associated with a family of nonstructural proteins termed sericin, which glue the fibroin brings together in forming the cocoon. The heavy and light chains of fibroin are linked by a disulfide bond at the C-terminus of the two subunits (see Takei, F., Kikuchi, Y., Kikuchi, A., Mizuno, S. and Shimura, K. (1987) 105 J. Cell Biol., 175-180; see also Tanaka, K., Mori, K. and Mizuno, S. 114 J. Biochem. (Tokyo), 1-4 (1993); Tanaka, K., Kajiyama, N., Ishikura, K., Waga, S., Kikuchi, A., Ohtomo, K., Takagi, T. and Mizuno, S., 1432 Biochim. Biophys. Acta., 92-103 (1999); Y Kikuchi, K Mori, S Suzuki, K Yamaguchi and S Mizuno, “Structure of the Bombyx mori fibroin light-chain-encoding gene: upstream sequence elements common to the light and heavy chain,” 110 Gene, 151-158 (1992)). The sericins are a high molecular weight, soluble glycoprotein constituent of silk which gives the stickiness to the material. These glycoproteins are hydrophilic and can be easily removed from cocoons by boiling in water.

In some embodiments, a silk solution is used to fabricate compositions of the present invention that contain fibroin proteins, essentially free of sericins. In some embodiments, silk solutions used to fabricate various compositions of the present invention contain the heavy chain of fibroin, but are essentially free of other proteins. In other embodiments, silk solutions used to fabricate various compositions of the present invention contain both the heavy and light chains of fibroin, but are essentially free of other proteins. In certain embodiments, silk solutions used to fabricate various compositions of the present invention comprise both a heavy and a light chain of silk fibroin; in some such embodiments, the heavy chain and the light chain of silk fibroin are linked via at least one disulfide bond. In some embodiments where the heavy and light chains of fibroin are present, they are linked via one, two, three or more disulfide bonds. Although different species of silk-producing organisms, and different types of silk, have different amino acid compositions, various fibroin proteins share certain structural features. A general trend in silk fibroin structure is a sequence of amino acids that is characterized by usually alternating glycine and alanine, or alanine alone. Such configuration allows fibroin molecules to self-assemble into a beta-sheet conformation. These “Alanine-rich” hydrophobic blocks are typically separated by segments of amino acids with bulky side-groups (e.g., hydrophilic spacers).

Silk fibroin materials explicitly exemplified herein were typically prepared from material spun by silkworm, Bombyx mori. Typically, cocoons are boiled in an aqueous solution of 0.02 M Na2C03, then rinsed thoroughly with water to extract the glue-like sericin proteins (this is also referred to as “degumming” silk). Extracted silk is then dissolved in a solvent, for example, LiBr (such as 9.3 M) solution at room temperature. A resulting silk fibroin solution can then be further processed for a variety of applications as described elsewhere herein.

In some embodiments, polymers of silk fibroin fragments can be derived by degumming silk cocoons at or close to (e.g., within 5% around) an atmospheric boiling temperature for at least about: 1 minute of boiling, 2 minutes of boiling, 3 minutes of boiling, 4 minutes of boiling, 5 minutes of boiling, 6 minutes of boiling, 7 minutes of boiling, 8 minutes of boiling, 9 minutes of boiling, 10 minutes of boiling, 11 minutes of boiling, 12 minutes of boiling, 13 minutes of boiling, 14 minutes of boiling, 15 minutes of boiling, 16 minutes of boiling, 17 minutes of boiling, 18 minutes of boiling, 19 minutes of boiling, 20 minutes of boiling, 25 minutes of boiling, 30 minutes of boiling, 35 minutes of boiling, 40 minutes of boiling, 45 minutes of boiling, 50 minutes of boiling, 55 minutes of boiling, 60 minutes or longer, including, e.g., at least 70 minutes, at least 80 minutes, at least 90 minutes, at least 100 minutes, at least 110 minutes, at least about 120 minutes or longer. As used herein, the term “atmospheric boiling temperature” refers to a temperature at which a liquid boils under atmospheric pressure.

As used herein, the phrase “silk fibroin fragments” refers to peptide chains or polypeptides having an amino acid sequence corresponding to fragments derived from silk fibroin protein or variants thereof. In the context of the present disclosure, silk fibroin fragments generally refer to silk fibroin peptide chains or polypeptides that are smaller than the naturally occurring full length silk fibroin counterpart, such that one or more of the silk fibroin fragments within a population or composition are less than 300 kDa. The provided silk fibroin fragments may be degummed under a specific condition (e.g., degumming time and atmospheric boiling temperature or a temperature ranging from 90° C. to 110° C.) to produce silk fibroin fragments having a desired molecular weight. In some embodiments, a silk solution may be produced having silk fibroin with a molecular weight that ranges from 3.5 kDa to 300 kDa, from 50 kDa to 120 kDa, or from 120 kDa to 300 kDa. In some embodiments, the molecular weight is at least 3.5 kDa, or at least 5 kDa, or at least 10 kDa, or at least 20 kDa, or at least 30 kDa, or at least 40 kDa, or at least 50 kDa, or at least 60 kDa, or at least 70 kDa, or at least 80 kDa, or at least 90 kDa, to less than 100 kDa, or less than 110 kDa, or less than 120 kDa, or less than 130 kDa, or less than 140 kDa, or less than 150 kDa, or less than 200 kDa, or less than 250 kDa, or less than 300 kDa. In some cases, the silk fibroin can be a low molecular weight silk fibroin, such as is described in WO 2014/145002, which is incorporated herein in its entirety by reference.

As the field of nanomedicine advances, concerns surrounding safety of the materials utilized have emerged. Inorganic nanoparticles such as metal-based particles have been used extensively and several have FDA approval for drug delivery, diagnostics, and imaging; however cytotoxicity concerns including cell membrane disruption, production of reactive oxygen species (ROS), DNA damage, and release of metal ions that affect protein function prevent their wider use. Similarly, lipid-based nanoparticles have had significant clinical success in recent years, but can induce oxidative stress, acidification of the cytosol, and thus inhibition of protein synthesis in vitro and have been reported to induce liver and lung damage in vivo. Some of the earliest polymers used to generate nanoparticles for in vivo testing, such as poly (methyl methacrylate) and polystyrene, were not biodegradable and have since been found to cause the release of proinflammatory cytokines, inducing local inflammation, increased ROS, and lactate dehydrogenase concentrations, as well as cell cycle arrest when these materials were assessed in vitro and vivo. In contrast, biodegradable polymeric nanoparticles, such as protein-based particles, have many intrinsic characteristics that make them good candidates for targeted drug delivery. Compared to conventional synthetic polymeric nanoparticles, protein-based particles can be cleaved by proteolytic enzymes and broken down into amino acids which are then metabolized or absorbed by the body. The proteins can also be chemically tailored to display cell-targeting ligands or other biomolecules of interest on their surface. They can be tuned to express a positive or negative surface charge by installing appropriate chemical blocks to influence cytotoxicity and therapeutic fate. Biodegradable protein-based nanoparticles can also be utilized to control the release profiles of drugs and avoid clearance by the reticuloendothelial system. Protein-based particles have already begun making their way into the clinic—Abraxane®, for example, is an albumin-bound particle form of paclitaxel that is widely used in the clinic and more albumin-bound particles are entering clinical trials, highlighting the growing use of protein-based nanoparticle systems.

In addition to degradability, size is an important parameter for the physical properties of nanoparticles, influencing adsorption rates, recognition by immune cells, travel through tight endothelial junctions, filtration by the spleen, among many other factors. In general, smaller particles (˜80 nm) circulate in the blood stream longer than larger particles (>200 nm). In cancer therapeutics, nanoparticles can exploit the enhanced permeation and retention effect (EPR), where the leaky vasculature of solid tumors and the weak lymphatic drainage synergistically encourage particle accumulation in target cells. In the endothelium of blood vessels of tumors, barrier distortion can result in pores, therefore nanoparticles should be smaller than these pores (generally <200 nm), but larger than 30 nm to exploit the EPR effect, although these size ranges will depend on the cell type and material. Size is a factor in drug release kinetics and mechanics, as different particle sizes could result in varying pharmacokinetics or mechanical behaviors when embedded in different matrices. In drug delivery or scaffold systems that require more controlled release or increased mechanical integrity, nanoparticles of varying sizes may be loaded with different therapeutics prior to casting in different matrices, or they could be simply used as a standalone structural component. This can be helpful in the case of bioprinting with silk, as silk-based bioinks must be highly concentrated/more viscous to avoid collapse of the printed object, which can cause clogging of the printer. By incorporating nanoparticles in the silk bioinks, lower concentrations of silk solution would be required to print structurally sound objects and prevent clogging. The nanoparticles could also be loaded with growth factors to enhance cell proliferation within these constructs. Additionally, in the case of silk microneedles, needles frequently break before penetrating the skin due to the needles often being hollow. Incorporation of silk microparticles may allow for filling in the needles and making them stronger as well as increased drug loading; however since the particles were too large, incorporation of them resulted in irregularly shaped microneedles. Using the smaller nanoparticles can fill in the needles and increase their mechanical integrity without loss of the desired shape.

Silk fibroin (hereafter referred to as silk), is biocompatible and degradable and has gained utility in drug delivery and nanoparticle research due to control of crystallinity which impacts degradation rates, chemical structure, and assembly into materials that stabilize therapeutics that are otherwise susceptible to denaturation. Various methods have been utilized to generate SNPs, including blending polymers such as silk-polyvinyl alcohol (PVA), spray drying, and nanoprecipitation among others.

Nanoprecipitation or desolvation is among the most popular methods of generating SNPs in the literature, and involves two miscible solutions, where the first solvent contains the polymer, and the second solvent does not (the precipitation solvent). This method involves the rapid dissolution of the polymer, which induces the precipitation of nanoparticles when the polymer solution is added to the precipitation solvent. This may occur due to the Marangoni effect, where the interfacial turbulence between solvent and nonsolvent govern particle formation. One limitation of the current nanoprecipitation method is the inability to precisely control the size of the resulting nanoparticles across a broad size range. In addition, the maintenance of a low polydispersity index (PDI) is desired in nanoparticle applications. Nanoprecipitation of natural biopolymers often produces particles greater than 100 nm in diameter. For example, recent work on nanoprecipitated particles prepared with naturally derived biopolymers have resulted in gelatin nanoparticles with sizes of 130-190 nm and 273 nm in a one-step and two-step fabrication method, chitosan particles with a size range of 200-700 nm with irregular particles formed at the larger sizes, and albumin nanoparticles with a size range of 90-450 nm with a broad PDI (0.02-0.8).

Nanoprecipitated SNPs have been generated with a 100 nm diameter using low molecular weight (MW) silk, and have been loaded with doxorubicin (DOX) to treat a human breast cancer cell line. These SNPs colocalized into lysosomes, thus, demonstrating potential for cancer treatments. The particles were also biocompatible in non-drug loaded formulations. However, a limitation to this method was the inability to produce SNPs across a broader size range while maintaining a low PDI. Investigation of the effect of stir rate (0, 200, and 400 rpm) during silk nanoparticle formation was also completed recently, and particles between 104-134 nm were generated using isopropanol as the nonsolvent. There have been limited studies to date to understand how to reproducibly control nanoprecipitated SNP size and PDI, yet the effect of size on cellular uptake and drug release is key. Additionally, previous methods to generate DOX-loaded SNPs only incubated pre-made particles in drug solutions to provide drug coatings (adsorption) on the particles, which limits protection from the surrounding environment, and prevents the addition of post-fabrication surface modifications.

This disclosure provides nanoprecipitated SNPs that can be reliably and reproducibly generated over a diverse size range from ˜45-250 nm while maintaining a low PDI (˜0.2-0.4). Significant control over size of the resulting SNPs may be achieved by changing silk properties (e.g., molecular weight, concentration) and reaction bath parameters (e.g., temperature, stir speed). In addition to size, the surface properties of these nanoparticles were altered by using pre-functionalized silk as starting precursor molecules or chemically appending appropriate pendant groups to the surface of the SNPs in a post-functionalization process. SNPs of different sizes may be successfully incubated with a cancer cell line (glioblastoma) as a model for cellular uptake and investigation of these particles for oncologic applications, and the entrapment of DOX in the SNPs is also disclosed.

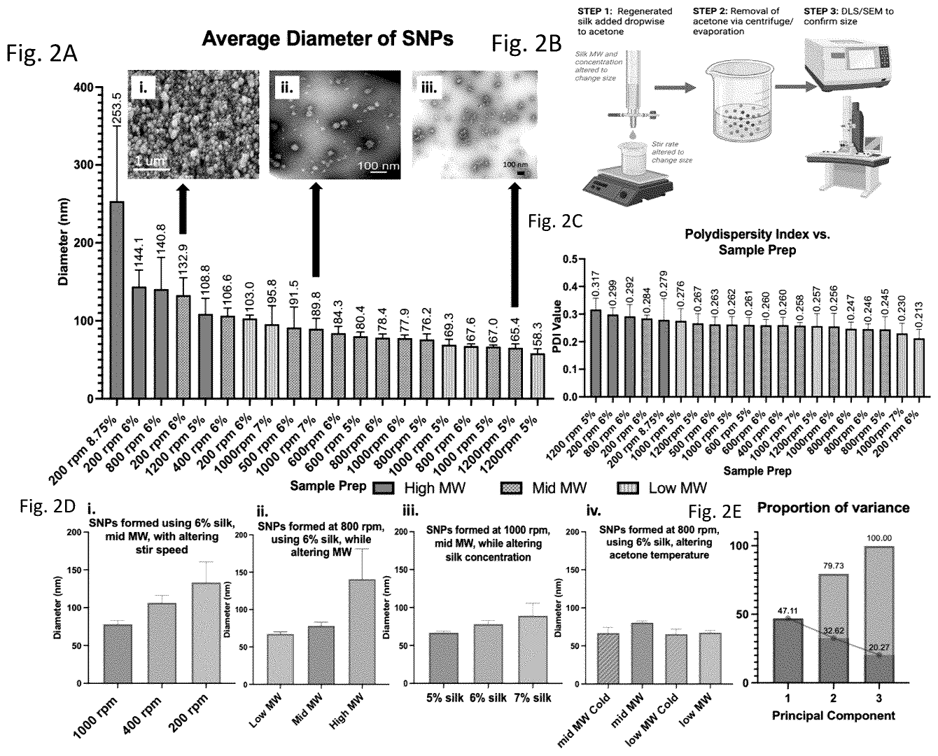

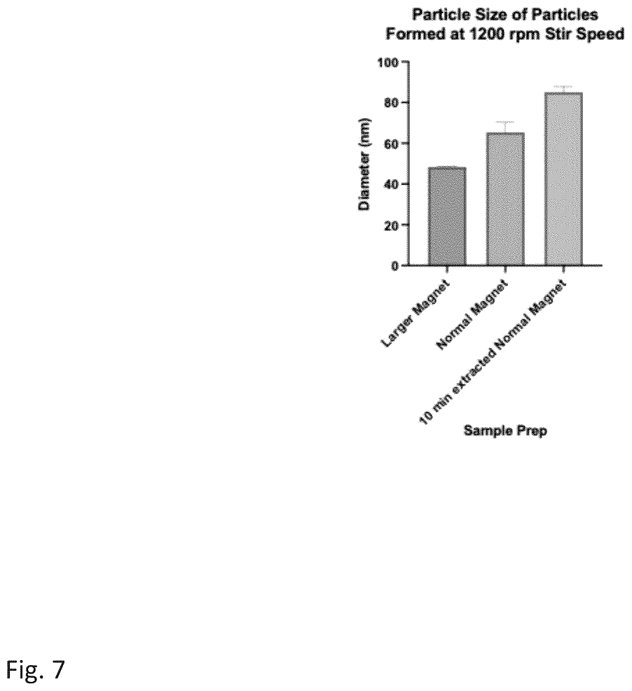

Controlling Size of SNPs and PDI of SNPs: SNPs of various sizes may be synthesized via the nanoprecipitation techniques disclosed herein (FIG. 2A and FIG. 2B). All particles generated had a PDI between ˜0.2-0.32 (FIG. 2C) which was independent of size. Decreasing stir speed (FIG. 2Di), while increasing silk molecular weight (FIG. 2Dii) and concentration (FIG. 2Diii) led to larger particle sizes, while decreasing silk concentration, molecular weight, and increasing stir speed resulted in smaller particle sizes. Further, when more vigorous magnetic stirring was used, thus increasing shear forces, smaller particles formed (FIG. 7). For example, when a magnetic stirrer is used with a stronger magnet (which would induce more rigorous shear forces on the particle), smaller particles are formed. When particles were formed in chilled (−20° C.) acetone, smaller particles generally formed (FIG. 2Div). From PCA analysis (FIG. 2E), stir speed of the acetone bath during nanoprecipitation of the particles accounted for 47.11%, the weight percent of silk for 32.62%, and molecular weight of the silk 20.27% of variance in particle diameter, respectively. Different molecular weights were achieved via extraction (boiling) times. For example, a 60-minute extraction yielded low MW (<171 kDa), a 30 min extraction yielded mid MW (31-268 kDa), and a 10 min extraction yielded high MW (171-460 kDa).

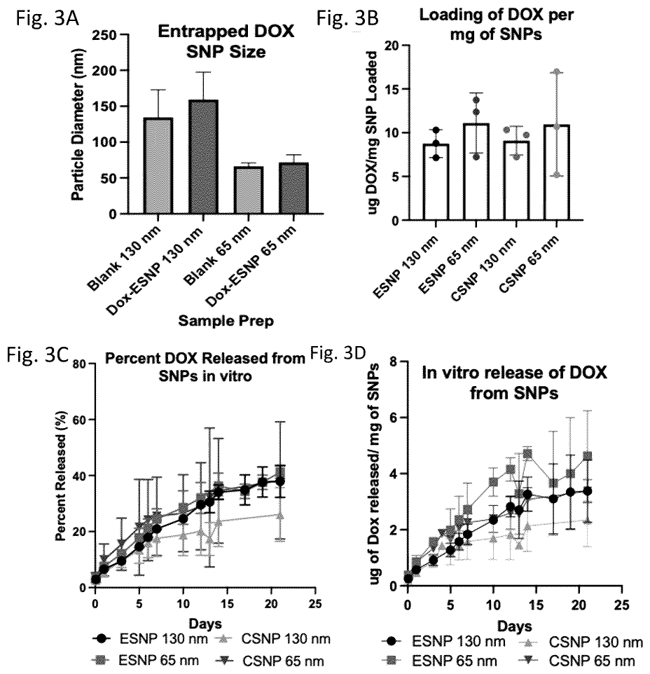

Doxorubicin-loaded SNPs: Doxorubicin pre-dissolved in silk solution prior to nanoprecipitation yielded particles of similar sizes as the unloaded particles, indicating that doxorubicin can be entrapped within SNPs and maintain similar particle size control as established in FIG. 2 (FIG. 3A). Differences in loading occurred when the same amount of doxorubicin (2 mg) was added to each batch of SNPs (FIG. 3B); smaller particles demonstrated higher amounts of DOX loaded per mg of SNPs (CSNP: 10.96±5.906 ug/mg, ESNP: 11.11±3.436 ug/mg) than larger particles (CSNP: 9.09±1.653 ug/mg, ESNP: 8.751±1.586 ug/mg). In both systems, particles released doxorubicin over 21 days (FIG. 3C, D). Particles formed using the smaller particle formulation (1,200 rpm, 5% w/v silk concentration, and mid MW) released more DOX per mg of SNPs. After the 21 days, 37.96%±5.7 and 41.22%±2.2 of DOX initially loaded had been released from the ESNP 130 nm and ESNP 65 nm samples, respectively, while 26.10%±9.6 and 38.26%±20.93 released from the CSNP 130 nm and CSNP 65 nm samples, respectively (FIG. 3C).

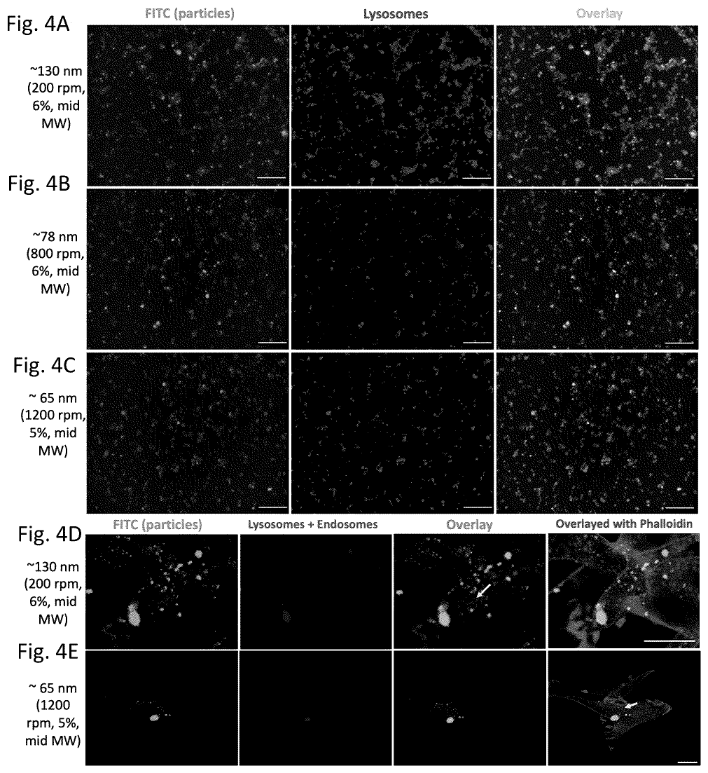

Cellular Uptake of SNPs of different sizes: For cellular uptake, the particles were taken up into glioblastoma cells after 4 h of incubation (FIG. 4A-C). In both live and fixed cellular uptake experiments, the particles colocalized to the lysosomes and/or endosomes (FIG. 4A-C colocalization=red/yellow, FIG. 4D,E colocalization=cyan); however nanoparticles also seemed to be located in areas that did not colocalize with the endosomes, lysosomes, or the cytoskeleton of the cells, which would have been indicated by phalloidin/FITC overlap (FIG. 4D,E, colocalization=yellow). This result suggests that there were particles escaping the endosomes or lysosomes but are still in intracellular locations. Confirmation of intracellular uptake via appropriate controls for live cell imaging was done by quenching any extracellular FITC using trypan blue. Confirmation of intracellular uptake for confocal, fixed cell imaging was accomplished via observations of areas that did not colocalize with phalloidin. Intracellular visualization of larger SNPs (130 nm) was more pronounced at the 4 h timepoint than for the smaller SNPs (78 nm or 65 nm) based on both live and fixed cell imaging.

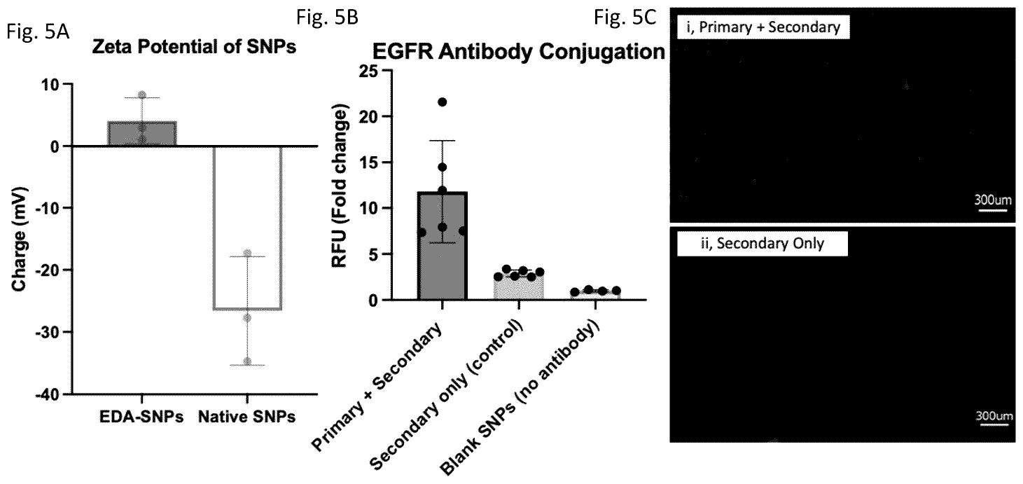

Modification of Materials Properties—Charge and Antibody Conjugation of SNPs: Unmodified SNPs have been reported to exhibit a zeta potential as low as −49 mV. By using EDA-modified silk, (See Hasturk O, Sahoo J K, Kaplan D L. Synthesis and Characterization of Silk Ionomers for Layer-by-Layer Electrostatic Deposition on Individual Mammalian Cells. Biomacromolecules. 2020; 21(7):2829-43.) nanoparticles were fabricated to express a positive surface charge (FIG. 5A), since EDA incorporation increased the primary amine content in silk. EDC coupling mechanisms have previously supported surface immobilization of antibodies on other silk-based heterogeneous material format like films; however here the goal was to couple antibodies to nanoprecipitated SNPs. Thus, a primary antibody was coupled to the SNPs using EDC/NHS carbodiimide coupling chemistry and the covalent incorporation of the antibody was validated using a fluorescent secondary antibody. In comparison to SNPs incubated in secondary antibody solution without the primary antibody conjugation (control), SNPs with the primary antibody previously conjugated showed significantly higher fluorescence intensity (FIG. 5B, C) to support the successful conjugation of the primary antibody to the surface of the SNPs. Any fluorescence expressed by the SNPs incubated in secondary antibody solution without the primary antibody conjugation (FIG. 5Cii) demonstrates nonspecific binding as a control. The same experiment was repeated for ESNPs, and it was found that while less antibody was able to be conjugated than the non-drug loaded particles, the anti-EGFR conjugated ESNPs showed significantly higher fluorescence intensity than the controls without inducing changes of DOX fluorescence by the SNPs from the conjugation process.

Silk particle size controlled by molecular weight, concentration, temperature, stir rate: Several variables (either precursor properties like MW and concentration or reaction bath condition (temperature, stirring speed) were altered to assess their impact on the size of the resulting nanoparticles. Silk is shear responsive and crystallizes when shear forces are applied. Additionally, water miscible organic monohydric solvents such as methanol and ethanol or polar solvents such as acetone increase the crystallinity in silk materials by accelerating beta-sheet structure formation.

We hypothesized that combining shear forces induced by increased magnetic stirring rate and maintaining exposure to organic solvents would increase the crystallization rate of silk, thus, resulting in smaller particles. Stirring also decreased silk chain aggregation, supporting a lower PDI. Further, silk is also temperature responsive and crystallizes more rapidly at higher temperatures. We hypothesized that cooling the acetone bath would result in slower silk aggregation, resulting in smaller particles than the process run at RT.

Silk nanoprecipitation is governed by the shift of water molecules away from the hydration shell of silk. Based on previous studies, we hypothesized that more efficient, and rapid “ripping” of the water molecules away from the silk hydration shell by magnetic stirring would generate smaller particles, and that this could be expanded with a broader range of stir speeds. Lower MW silk was found to generate smaller sized particles. We also hypothesized that lower concentrations of silk would result in the formation of smaller particles, as decreasing silk content while keeping the acetone volumes constant might result in reduced exposure of silk hydration shells to the acetone. Altering SNP features: Protein-based nanoparticles have many characteristics that are advantageous for targeted drug delivery. Accessible chemistries, imparted by presence of many chemically active amino acids, is one of them. They can be easily chemically modified using many established routes to display cell-targeting ligands on their surface and can be tuned to display a positive or negative surface charge by functionalization with appropriate ligands. In vivo, these surface charges will change due to the protein corona formed around the particles, which describes the layer of proteins in physiological fluids that assemble on the surface of nanoparticles. This protein layer is governed by the physicochemical properties of the nanoparticle, such as surface charge and hydrophobicity. With protein-based particles like silk, surface charge and degree of hydrophobicity can be readily tuned, allowing for potential future control of the protein corona in vivo. Tuning surface charge of SNPs is advantageous for intracellular delivery of active pharmaceutical agents, as charge can affect the serum proteins absorbed onto the surface of the particles and thus cellular uptake, and as cells are slightly negatively surface charged. Additionally, positively charged particles can support coatings of negatively charged polymers such as alginate and negatively charged antigens or drugs, to bind to the surface of the nanoparticles via electrostatic interactions.

Prior to this research, a significant limitation with silk nanoparticles formed via nanoprecipitation was a lack of size control across a broad size range while maintaining a low PDI and morphology. This was also the case for other biopolymers, as mentioned (gelatin, chitosan). In this disclosure, we have demonstrated the expansion of processing parameters to generate reproducible NPs with a wider range of specific sizes using nanoprecipitation while also maintaining a tight PDI. This control and insight should support new opportunities to utilize such defined and functionalized SNPs in new targeting applications.

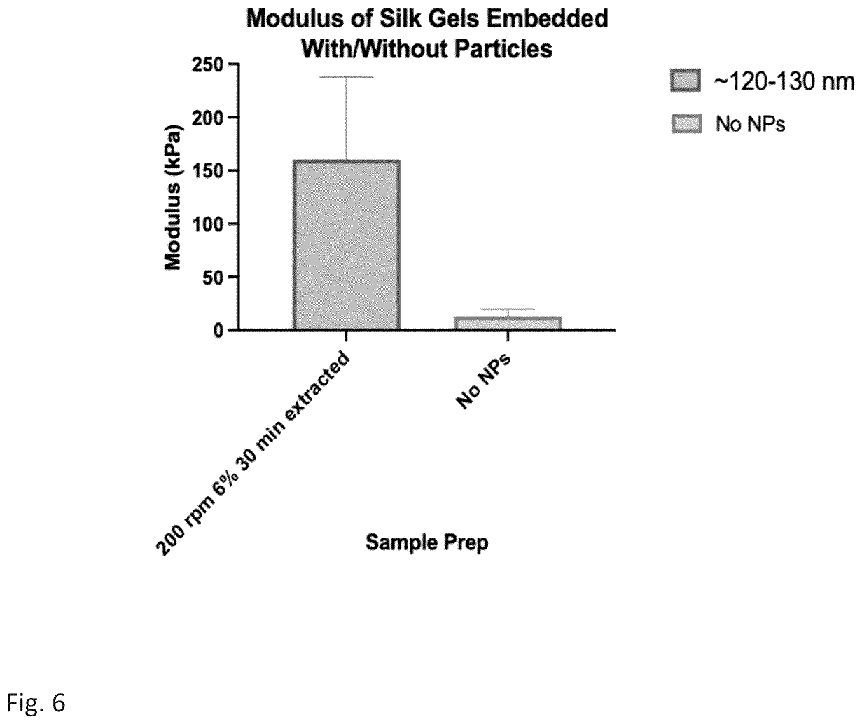

Understanding how size affects drug release kinetics is critical, and here the release of doxorubicin from the 65 nm SNPs was more rapid in comparison to the 130 nm SNPs, a difference that may be due to the higher surface-to-volume-ratio of the smaller particles, which would induce more rapid release of the DOX out of the smaller particles. We also showed differences in loading between the smaller and larger particles, possibly due to the silk content used to formulate the particles, as the 65 nm formulation utilized 5% w/v silk, and the 130 nm formulation utilized 6% w/v silk. Additionally, there was increased variability in the loading and the percent of DOX released in the CSNPs over time in comparison to the ESNPs, suggesting that entrapment of DOX rather than coatings (adsorption) leads to a more reproducible release profile and loading. As described herein, an advantage of polymeric nanoparticles is the ease of attaching cell-targeting ligands on their surface, yet the post-processing conditions required to attach these ligands can lead to the loss of drug coatings. Drug coatings may block the targeting ligands from adhering to the surface of the SNPs. Drug coatings on NPs can also result in premature loss of drug before reaching the target site in vivo, which in the case of chemotherapy, would cause harm to healthy tissues and insufficient cell death in the target (cancerous) tissue. Entrapment of doxorubicin within SNPs, rather than as a coating, therefore, has the potential to protect the chemotherapy drugs during the post-processing conditions. Although there was decreased antibody conjugation demonstrated in ESNPs relative to non-drug loaded SNPs, we show that anti-EGFR was still able to attach successfully in comparison to controls. The decreased antibody conjugation to the ESNPs in comparison to the SNPs may be due to DOX-silk interactions present on the surface of ESNPs decreasing the available area for the conjugation to occur in comparison to non-drug loaded particles. Additionally, other methods to produce SNPs with chemotherapy drugs entrapped within the particles mostly utilize sizes larger than 300 nm, thus, rendering them less useful for cellular uptake. Therefore, we successfully generated SNPs where the DOX was added to the silk prior to generating the particles to efficiently entrap the drug, while not significantly altering the particle sizes and resulting in sustained in vitro release over 20 days. While this release profile would not be observed at a cellular level as the particles will likely be destroyed in the lysosomes of the cells resulting in immediate drug release, this sustained release profile could be useful for other drug delivery systems where SNPs are embedded within other materials such as hydrogels or spongy scaffolds. Further, and referring now to FIG. 6, after embedding ˜4 mg of nanoparticles into silk fibroin hydrogels, it was found that nanoparticles increased the mechanical integrity of hydrogels, and the hydrogels gelled more readily than silk gels without nanoparticles, demonstrating the potential applications of using nanoparticles in localized injectable drug delivery systems, bone cements, or bioprinting applications.

For internalization of nanoparticles by the cells, endocytic mechanisms (pinocytosis, endocytosis) depend on size and surface properties. After internalization, nanoparticles are typically transported from the early endosome to the late endosome, and then to the lysosomes to be disrupted. For delivery of mRNA, nanoparticles must escape the endosome or lysosome and enter the cytoplasm to be effective; in contrast, accumulation in lysosomes is the goal for cancer treatments related to cell death by causing rupture of the acidic lysosome and pH-driven release of chemotherapy from the particles. We show that SNPs fabricated using methods to produce 65, 78, and 130 nm are taken up in GBM cells after 4 h of incubation, and SNPs mostly colocalize with the lysosomes, demonstrating potential of the SNPs being used for lysosomal delivery of cargos. Interestingly in all trialed particle sizes, some SNPs were observed in areas of the cells that were not the lysosomes or endosomes, showing the potential of these particle systems to be utilized for cytosolic delivery with improved engineering to specifically deliver the particles to the cytoplasm. We also show that the 130 nm SNPs have a higher degree of internalization at 4 h than the 65 nm SNPs.

Nanocarriers for drug delivery to the central nervous system (CNS) remains a major goal towards the treatment of CNS related diseases, such as glioblastoma multiforme (GBM), the most common primary CNS tumor in adults. Tumors often develop drug resistance, while a major contributor to poor patient survival is the challenge of delivering therapeutics across the blood brain barrier (BBB). The role of the BBB is to restrict entry of substances between the peripheral circulation and the CNS, thus, generally only lipophilic drugs with a molecular weight <500 Da can cross the BBB, ruling out the majority of potential drug candidates. Using nanotechnology to deliver various active pharmaceutical ingredients (APIs) to the brain, in particular GBM, has gained considerable research focus in the last few decades, as it has the potential to target specific areas of the brain and reduce adverse side effects that are associated with off-target API distribution. Thus, we conjugated anti-EGFR to the surface of SNPs, as EGFR gene amplification and overexpression is seen in 40-50% of GBMs making it a promising target for future use, and EGFR-specific antibodies cetuximab and panitumumab are widely used in metastatic colorectal cancers already. Nanotechnology can also be utilized for delivering APIs that normally do not pass the BBB into the brain, such as doxorubicin, which has been reported to have superior cytotoxic effects with GBM cell lines over the currently used BBB penetrable drug, temozolomide. The investigation of nanoprecipitated SNPs for the potential treatment of GBM, based on lysosomal uptake in GBM cells, successful doxorubicin entrapment, and conjugation of anti-EGFR suggest potential for future use of these particles for local delivery after maximal tumor resection, or potentially even BBB penetration and GBM targeting with further optimization. Future work will assess the behavior of these functionalized particles in cellular studies with protein corona delineation. Conclusions: Methods to control the size and surface characteristics of nanoprecipitated SNPs were investigated to provide improved control of size and dispersity. In addition, doxorubicin loaded SNPs were evaluated to show potential utility in sustained release for cancer drug delivery. New understanding of the cellular uptake of these particles into cancer cells was gained using GBM cells. The disclosed new protein nanoparticles with tight control of size, charge and delivery options should propel further studies into their utility in a range of biomedical needs. These needs may include targeted GBM chemotherapy treatment (as seen in this work), vaccines, infection, pain management, bone disease and regeneration, and other medical conditions. Overall, biocompatible and slowly degrading biomaterial systems like those disclosed here offer many areas of potential impact into the future as targeted functionalized versions are pursued.

An example method disclosed herein includes adding a silk solution dropwise into a volatile solvent that is miscible with water, thereby forming a precipitate-bearing solution. In embodiments, the silk solution contains silk fibroin, such as in an amount by weight of at least 2%, at least 3%, at least 5%, at least 6%, or at least 7%, and at most 25%. The precipitate-bearing solution may include the organic solvent in an amount of at least 75% (v/v). The volatile solvent may be acetone, ether, an alcohol or other solvents having comparable miscibility and/or boiling points.

Shear forces may be applied to the precipitate-bearing solution, such as by stirring. For example, a magnetic stir bar may be used to stir the silk solution. In some embodiments, variation of parameters related to the magnetic bar, such as its size and/or magnetic strength, may have an impact on shear forces applied and corresponding particle sizes achieved. The stirring may continue for a length of time sufficient to achieve evaporation of at least 95% of the volatile solvent, thereby producing a population of silk fibroin nanoparticles in water. Stirring may be performed at a temperature of between a freezing point of the precipitate-bearing solution and 60° C., such as at −20° C.

The population of silk fibroin nanoparticles in water may have a polydispersity index (PDI) of 0.35 or less, including but not limited to, a PDI of 0.330 or less, 0.325 or less, 0.315 or less, 0.310 or less, 0.30 or less, 0.275 or less, 0.250 or less, 0.225 or less, or 0.200 or less. The population of silk fibroin nanoparticles may be sonicated, in embodiments.

In embodiments, the silk solution may include an active agent, such as any of the active agents disclosed herein. Optionally, the active agent may be doxorubicin. The presence of the active agent in the silk solution results in the active agent being embedded within the population of silk fibroin nanoparticles.

In some embodiments, the method may include crosslinking individual silk fibroin molecules within individual silk fibroin nanoparticles. Crosslinking may be achieved by adding an enzymatic crosslinker to the population of silk fibroin nanoparticles, such as glutaraldehyde, transglutaminase, or peroxidase.

In some embodiments, the method may include surface modifying the population of silk fibroin nanoparticles, such as by affixing antibodies to the population of silk fibroin nanoparticles.

In some embodiments, the method may include adjusting surface charge of the population of silk fibroin nanoparticles.

An example method of administering silk fibroin nanoparticles may include administering a first plurality of silk fibroin nanoparticles having an average diameter below a predetermined size threshold and a polydispersity index of 0.35 or less, including but not limited to, a PDI of 0.330 or less, 0.325 or less, 0.315 or less, 0.310 or less, 0.30 or less, 0.275 or less, 0.250 or less, 0.225 or less, or 0.200 or less.

Example methods disclosed herein may be useful in tuning silk nanoparticle sizes, such as to generated smaller or larger particle sizes, by selecting one or more variables of a nanoprecipitation process. An example method of tuning silk nanoparticle size may include obtaining a silk solution including silk fibroin of a selected molecular weight and a selected concentration. The silk solution may be added dropwise into a volatile solvent that is miscible with water, thereby forming a precipitate-bearing solution. The precipitate-bearing solution may include the organic solvent in an amount of at least 75% (v/v). Shear forces may be applied to the precipitate-bearing solution, the applying continuing for a length of time sufficient to achieve evaporation of at least 95% of the volatile solvent, thereby producing a population of silk fibroin nanoparticles in water having a polydispersity index (PDI) of 0.35 or less, including but not limited to, a PDI of 0.330 or less, 0.325 or less, 0.315 or less, 0.310 or less, 0.30 or less, 0.275 or less, 0.250 or less, 0.225 or less, or 0.200 or less. For example, decreasing stir speed while increasing silk molecular weight and concentration may result in larger silk nanoparticle sizes. In another example, decreasing silk concentration, decreasing molecular weight, and increasing stir speed may result in smaller silk nanoparticle sizes. Data presented throughout this Specification and in the Figures, particularly FIG. 2A-E, demonstrate the relationship of these parameters on the average diameter of SNPs. The selected molecular weight may be selected to produce a population of silk fibroin nanoparticles of a predetermined size range. The selected concentration may be selected to produce a population of silk fibroin nanoparticles of a predetermined size range. The applying may be performed by stirring, such as with a magnetic stir bar as described elsewhere herein, at a stir speed selected to produce a population of silk fibroin nanoparticles of a predetermined size range. The applying may be performed at a temperature selected to produce a population of silk fibroin nanoparticles of a predetermined size range, such as a temperature between a freezing point of the precipitate-bearing solution and 60° C.

In embodiments, the silk solution may include silk fibroin in an amount by weight of at least 2%, at least 3%, at least 5%, at least 6%, or at least 7%, and at most 25%. In embodiments, the dropwise addition may be at a drop length of at least 6 cm, at least 7 cm, at least 8 cm, at most 6 cm, at most 7 cm, or at most 8 cm. The dropwise addition may be at a drop rate of most 8 drops/minute, at most 9 drops/min, at most 10 drops/min, at least 8 drops/min, at least 9 drops/min, or at least 10 drops/min.

In embodiments, the silk solution may include an active agent as disclosed herein. The active agent may optionally be doxorubicin. The presence of the active agent in the silk solution results in the active agent being embedded within the population of silk fibroin nanoparticles. The volatile solvent may be acetone, ether, an alcohol or other solvents having comparable miscibility and/or boiling points.

The methods disclosed herein may further include sonicating the population of silk fibroin nanoparticles or crosslinking individual silk fibroin molecules within individual silk fibroin nanoparticles. Crosslinking may be achieved by adding an enzymatic crosslinker to the population of silk fibroin nanoparticles, wherein the enzymatic crosslinker is optionally glutaraldehyde, transglutaminase, or peroxidase.

The methods disclosed herein may further include surface modifying the population of silk fibroin nanoparticles, such as by affixing antibodies to the population of silk fibroin nanoparticles. The methods disclosed herein may further include adjusting surface charge of the population of silk fibroin nanoparticles.

Compositions may be made by any of the methods disclosed herein. In some embodiments, the composition is a hydrogel having the population of silk fibroin nanoparticles embedded therein. The hydrogel may be a silk fibroin hydrogel.

According to various embodiments, a variety of functionalizing agents may be used with the silk-containing embodiments described herein (e.g., silk membrane, silk composition, silk matrix, silk foam, silk microsphere, etc.). It should be understood that the examples herein may recite one or a few silk-containing embodiments but are applicable to any silk-containing embodiment, as applicable. Also, it should be understood that functionalizing agents may be used to pre-functionalize silk embodiments, or may be used in a post-functionalization process. In some embodiments, a functionalizing agent may be any compound or molecule that facilitates the attachment to and/or development (e.g., growth) of one or more endothelial cells on a silk membrane. In some embodiments, a functionalizing agent may be any compound or molecule that facilitates the attachment and/or development (e.g., growth) of one or more megakaryocytes and/or hematopoietic progenitor cells on a silk matrix and/or silk membrane. In some embodiments, a functionalizing agent may be or comprise an agent suitable for facilitating the production of one or more of white blood cells and red blood cells.

In some embodiments, a functionalizing agent may be or comprise a cell attachment mediator and/or an extracellular matrix protein, for example: collagen (e.g., collagen type I, collagen type III, collagen type IV or collagen type VI), elastin, fibronectin, vitronectin, laminin, fibrinogen, von Willebrand factor, proteoglycans, decorin, perlecan, nidogen, hyaluronan, and/or peptides containing known integrin binding domains e.g. “RGD” integrin binding sequence, or variations thereof, that are known to affect cellular attachment.

In some embodiments, a functionalizing agent may be any soluble molecule produced by endothelial cells. Non-limiting examples include fibroblast growth factor-1 (FGF1) and vascular endothelial growth factors (VEGF).

According to some embodiments, a plurality of functionalizing agents may be used. For example, in some embodiments wherein production of platelets is desired, provided compositions may comprise the use of laminin, fibronectin and/or fibrinogen, and type IV collagen in order to facilitate the attachment and growth of endothelial cells on a silk membrane (e.g., a porous silk membrane) and/or attachment of megakaryocytes to a silk matrix.

In some embodiments, a functionalizing agent may be embedded or otherwise associated with a silk membrane and/or silk matrix such that at least a portion of the functionalizing agent is surrounded by a silk membrane and/or silk matrix as contrasted to a functionalizing agent simply being positioned along the surface of a silk membrane and/or silk matrix. In some embodiments, a functionalizing agent is distributed along and/or incorporated in substantially the entire surface area of a silk membrane/silk wall. In some embodiments, a functionalizing agent is distributed and/or incorporated only at one or more discrete portions of a silk membrane/wall and/or silk matrix. In some embodiments, a functionalizing agent is distributed in and/or along at least one of the lumen-facing side of a silk wall and the matrix-facing side of a silk wall.

According to various embodiments, any application-appropriate amount of one or more functionalizing agents may be used. In some embodiments, the amount of an individual functionalizing agent may be between about 1 μg/ml and 1,000 μg/ml (e.g., between about 2 and 1,000, 5 and 1,000, 10 and 1,000, 10 and 500, 10 and 100 μg/ml). In some embodiments, the amount of an individual functionalizing agent may be at least 1 μg/ml (e.g., at least 5, 10, 15, 20 25, 50, 100, 200, 300 400, 500, 600, 700, 800, or 900 μg/ml). In some embodiments, the amount of an individual functionalizing agent is at most 1,000 μg/ml (e.g., 900, 800, 700, 600, 500, 400, 300 200, 100, 90, 80, 70, 60, 50, 40, 30, 20, 10, or 5 μg/ml).

In some aspects, the composition comprises one or more sensing agents, such as a sensing dye. The sensing agents/sensing dyes are environmentally sensitive and produce a measurable response to one or more environmental factors. In some aspects, the environmentally—sensitive agent or dye may be present in the composition in an effective amount to alter the composition from a first chemical-physical state to a second chemical-physical state in response to an environmental parameter (e.g., a change in pH, light intensity or exposure, temperature, pressure or strain, voltage, physiological parameter of a subject, and/or concentration of chemical species in the surrounding environment) or an externally applied stimulus (e.g., optical interrogation, acoustic interrogation, and/or applied heat). In some cases, the sensing dye is present to provide one optical appearance under one given set of environmental conditions and a second, different optical appearance under a different given set of environmental conditions. Suitable concentrations for the sensing agents described herein can be the concentrations for the colorants and additives described elsewhere herein. A person having ordinary skill in the chemical sensing arts can determine a concentration that is appropriate for use in a sensing application of the inks described herein.

In some aspects, the first and second chemical-physical state may be a physical property of the composition, such as mechanical property, a chemical property, an acoustical property, an electrical property, a magnetic property, an optical property, a thermal property, a radiological property, or an organoleptic property. Exemplary sensing dyes or agents include, but are not limited to, a pH sensitive agent, a thermal sensitive agent, a pressure or strain sensitive agent, a light sensitive agent, or a potentiometric agent.

Exemplary pH sensitive dyes or agents include, but are not limited to, cresol red, methyl violet, crystal violet, ethyl violet, malachite green, methyl green, 2-(p-dimethylaminophenylazo) pyridine, paramethyl red, metanil yellow, 4-phenylazodiphenylamine, thymol blue, metacresol purple, orange IV, 4-o-Tolylazo-o-toluindine, quinaldine red, 2,4-dinitrophenol, erythrosine disodium salt, benzopurpurine 4B, N,N-dimethyl-p-(m-tolylazo) aniline, p-dimethylaminoazobenene, 4,4′-bis(2-amino-1-naphthylazo)-2,2′-stilbenedisulfonic acid, tetrabromophenolphthalein ethyl ester, bromophenol blue, Congo red, methyl orange, ethyl orange, 4-(4-dimethylamino-1-naphylazo)-3-methoxybenesulfonic acid, bromocresol green, resazurin, 4-phenylazo-1-napthylamine, ethyl red 2-([-dimethylaminophenyazo) pyridine, 4-(p-ethoxypehnylazo)-m-phenylene-diamine monohydrochloride, resorcin blue, alizarin red S, methyl red, propyl red, bromocresol purple, chlorophenol red, p-nitrophenol, alizarin 2-(2,4-dinitrophenylazo) 1-napthol-3,6-disulfonic acid, bromothymol blue, 6,8-dinitro-2,4-(1H) quinazolinedione, brilliant yellow, phenol red, neutral red, m-nitrophenol, cresol red, turmeric, metacresol purple, 4,4′-bis(3-amino-1-naphthylazo)-2,2′-stilbenedisulfonic acid, thymol blue, p-naphtholbenzein, phenolphthalein, o-cresolphthalein, ethyl bis(2,4-dimethylphenyl) ethanoate, thymolphthalein, nitrazine yellow, alizarin yellow R, alizarin, p-(2,4-dihydroxyphenylazo) benzenesulfonic acid, 5,5′-indigodisulfonic acid, 2,4,6-trinitrotoluene, 1,3,5-trinitrobenezne, and clayton yellow.

Exemplary light responsive dyes or agents include, but are not limited to, photochromic compounds or agents, such as triarylmethanes, stilbenes, azasilbenes, nitrones, fulgides, spiropyrans, napthopyrans, spiro-oxzines, quinones, derivatives and combinations thereof.

Exemplary potentiometric dyes include, but are not limited to, substituted amiononaphthylehenylpridinium (ANEP) dyes, such as di-4-ANEPPS, di-8-ANEPPS, and N-(4-Sulfobutyl)-4-(6-(4-(Dibutylamino)phenyl)hexatrienyl)Pyridinium (RH237).

Exemplary temperature sensitive dyes or agents include, but are not limited to, thermochromic compounds or agents, such as thermochromic liquid crystals, leuco dyes, fluoran dyes, octadecylphosphonic acid.

Exemplary pressure or strain sensitive dyes or agents include, but are not limited to, spiropyran compounds and agents.

Exemplary chemi-sensitive dyes or agents include, but are not limited to, antibodies such as immunoglobulin G (IgG) which may change color from blue to red in response to bacterial contamination.

In some aspects, the compositions comprise one or more additive, dopant, or biologically active agent suitable for a desired intended purpose. In some aspects, the additive or dopant may be present in the composition in an amount effective to impart an optical or organoleptic property to the composition. Exemplary additives or dopants that impart optical or organoleptic properties include, but are not limited to, dyes/pigments, flavorants, aroma compounds, granular or fibrous fillers.

Additionally or alternatively, the additive, dopant, or biologically active agent may be present in the composition in an amount effective to “functionalize” the composition to impart a desired mechanical property or added functionality to the composition. Exemplary additive, dopants, or biologically active agent that impart the desired mechanical property or added functionality include, but are not limited to: environmentally sensitive/sensing dyes; active biomolecules; conductive or metallic particles; inorganic particles drugs (e.g., antibiotics, small molecules or low molecular weight organic compounds); proteins and fragments or complexes thereof (e.g., enzymes, antigens, antibodies and antigen-binding fragments thereof); cells and fractions thereof (viruses and viral particles; prokaryotic cells such as bacteria; eukaryotic cells such as mammalian cells and plant cells; fungi).

In some aspects, the additive or dopant comprises a flavoring agent or flavorant.

Exemplary flavorants include ester flavorants, amino acid flavorants, nucleic acid flavorants, organic acid flavorants, and inorganic acid flavorants, such as, but not limited to, diacetyl, acetylpropionyl, acetoin, isoamyl acetate, benzaldehyde, cinnamaldehyde, ethyl propionate, methyl anthranilate, limonene, ethyl decadienoate, allyl hexanoate, ethyl maltol, ethylvanillin, methyl salicylate, manzanate, glutamic acid salts, glycine salts, guanylic acids salts, inosinic acid salts, acetic acid, ascorbic acid, citric acid, fumaric acid, lactic acid, malic acid, phosphoric acid, tartaric acid, derivatives, and mixtures thereof.

In some aspects, the additive or dopant comprises an aroma compound. Exemplary aroma compounds include ester aroma compounds, terpene aroma compounds, cyclic terpenes, and aromatic aroma compounds, such as, but not limited to, geranyl acetate, methyl formate, metyl acetate, methyl propionate, methyl butyrate, ethyl acetate, ethyl butyrate, isoamyl acetate, pentyl butrate, pentyl pentanoate, octyl acetate, benzyl acetate, methyl anthranilate, myrecene, geraniol, nerol, citral, cironellal, cironellol, linalool, nerolidol, limonene, camphor, menthol, carone, terpineol, alpha-lonone, thujone, eucalyptol, benzaldehyde, eugenol, cinnamaldehyde, ethyl maltol, vanillin, anisole, anethole, estragole, thymol.

In some aspects, the additive or dopant comprises a colorant, such as a dye or pigment. In some aspects, the dye or pigment imparts a color or grayscale to the composition. The colorant can be different than the sensing agents and/or sensing dyes below. Any organic and/or inorganic pigments and dyes can be included in the inks. Exemplary pigments suitable for use in the present disclosure include International Color Index or C.I. Pigment Black Numbers 1, 7, 1 1 and 31, C.I. Pigment Blue Numbers 15, 15:1, 15:2, 15:3, 15:4, 15:6, 16, 27, 29, 61 and 62, C.I. Pigment Green Numbers 7, 17, 18 and 36, C.I. Pigment Orange Numbers 5, 13, 16, 34 and 36, C.I. Pigment Violet Numbers 3, 19, 23 and 27, C.I. Pigment Red Numbers 3, 17, 22, 23, 48:1, 48:2, 57:1, 81:1, 81:2, 81:3, 81:5, 101, 114, 122, 144, 146, 170, 176, 179, 181, 185, 188, 202, 206, 207, 210 and 249, C.I. Pigment Yellow Numbers 1, 2, 3, 12, 13, 14, 17, 42, 65, 73, 74, 75, 83, 30, 93, 109, 110, 128, 138, 139, 147, 142, 151, 154 and 180, D&C Red No. 7, D&C Red No. 6 and D&C Red No. 34, carbon black pigment (such as Regal 330, Cabot Corporation), quinacridone pigments (Quinacridone Magenta (228-0122), available from Sun Chemical Corporation, Fort Lee, N.J.), diarylide yellow pigment (such as AAOT Yellow (274-1788) available from Sun Chemical Corporation); and phthalocyanine blue pigment (such as Blue 15:3 (294-1298) available from Sun Chemical Corporation). The classes of dyes suitable for use in present invention can be selected from acid dyes, natural dyes, direct dyes (either cationic or anionic), basic dyes, and reactive dyes. The acid dyes, also regarded as anionic dyes, are soluble in water and mainly insoluble in organic solvents and are selected, from yellow acid dyes, orange acid dyes, red acid dyes, violet acid dyes, blue acid dyes, green acid dyes, and black acid dyes. European Patent 0745651, incorporated herein by reference, describes a number of acid dyes that are suitable for use in the present disclosure. Exemplary yellow acid dyes include Acid Yellow 1 International Color Index or C.I. 10316); Acid Yellow 7 (C.I. 56295); Acid Yellow 17 (C.I. 18965); Acid Yellow 23 (C.I. 19140); Acid Yellow 29 (C.I. 18900); Acid Yellow 36 (C.I. 13065); Acid Yellow 42 (C.I. 22910); Acid Yellow 73 (C.I. 45350); Acid Yellow 99 (C.I. 13908); Acid Yellow 194; and Food Yellow 3 (C.I. 15985). Exemplary orange acid dyes include Acid Orange 1 (C.I. 13090/1); Acid Orange 10 (C.I. 16230); Acid Orange 20 (C.I. 14603); Acid Orange 76 (C.I. 18870); Acid Orange 142; Food Orange 2 (C.I. 15980); and Orange B.

Exemplary red acid dyes include Acid Red 1. (C.I. 18050); Acid Red 4 (C.I. 14710); Acid Red 18 (C.I. 16255), Acid Red 26 (C.I. 16150); Acid Red 2.7 (C.I. as Acid Red 51 (C.I. 45430, available from BASF Corporation, Mt. Olive, N.J.) Acid Red 52 (C.I. 45100); Acid Red 73 (C.I. 27290); Acid Red 87 (C. I. 45380); Acid Red 94 (C.I. 45440) Acid Red 194; and Food Red 1 (C.I. 14700). Exemplary violet acid dyes include Acid Violet 7 (C.I. 18055); and Acid Violet 49 (C.I. 42640). Exemplary blue acid dyes include Acid Blue 1 (C.I. 42045); Acid Blue 9 (C.I. 42090); Acid Blue 22 (C.I. 42755); Acid Blue 74 (C.I. 73015); Acid Blue 93 (C.I. 42780); and Acid Blue 158A (C.I. 15050). Exemplary green acid dyes include Acid Green 1 (C.I. 10028); Acid Green 3 (C.I. 42085); Acid Green 5 (C.I. 42095); Acid Green 26 (C.I. 44025); and Food Green 3 (C.I. 42053). Exemplary black acid dyes include Acid Black 1 (C.I. 20470); Acid Black 194 (Basantol® X80, available from BASF Corporation, an azo/1:2 CR-complex.

Exemplary direct dyes for use in the present disclosure include Direct Blue 86 (C.I. 74180); Direct Blue 199; Direct Black 168; Direct Red 253; and Direct Yellow 107/132 (C.I. Not Assigned).

Exemplary natural dyes for use in the present disclosure include Alkanet (C.I. 75520, 75530); Annafto (C.I. 75120); Carotene (C.I. 75130); Chestnut; Cochineal (C.I.75470); Cutch (C.I. 75250, 75260); Divi-Divi; Fustic (C.I. 75240); Hypernic (C.I. 75280); Logwood (C.I. 75200); Osage Orange (C.I. 75660); Paprika; Quercitron (C.I. 75720); Sanrou (C.I. 75100); Sandal Wood (C.I. 75510, 75540, 75550, 75560); Sumac; and Tumeric (C.I. 75300). Exemplary reactive dyes for use in the present disclosure include Reactive Yellow 37 (monoazo dye); Reactive Black 31 (disazo dye); Reactive Blue 77 (phthalo cyanine dye) and Reactive Red 180 and Reactive Red 108 dyes. Suitable also are the colorants described in The Printing Ink Manual (5th ed., Leach et al. eds. (2007), pages 289-299. Other organic and inorganic pigments and dyes and combinations thereof can be used to achieve the colors desired.

In addition to or in place of visible colorants, compositions provided herein can contain ETV fluorophores that are excited in the ETV range and emit light at a higher wavelength (typically 400 nm and above). Examples of ETV fluorophores include but are not limited to materials from the coumarin, benzoxazole, rhodamine, napthalimide, perylene, benzanthrones, benzoxanthones or benzothia-xanthones families. The addition of a UV fluorophore (such as an optical brightener for instance) can help maintain maximum visible light transmission. The amount of colorant, when present, generally is between 0.05% to 5% or between 0.1% and 1% based on the weight of the composition.

For non-white compositions, the amount of pigment/dye generally is present in an amount of from at or about 0.1 wt % to at or about 20 wt % based on the weight of the composition. In some applications, a non-white ink can include 15 wt % or less pigment/dye, or 10 wt % or less pigment/dye or 5 wt % pigment/dye, or 1 wt % pigment/dye based on the weight of the composition. In some applications, a non-white ink can include 1 wt % to 10 wt %, or 5 wt % to 15 wt %, or 10 wt % to 20 wt % pigment/dye based on the weight of the composition. In some applications, a non-white ink can contain an amount of dye/pigment that is 1 wt %, 2 wt %, 3 wt %, 4 wt %, 5%, 6 wt %, 7 wt %, 8 wt %, 9 wt %, 10 wt %, 11 wt %, 12 wt %, 13 wt %, 14 wt %, 15%, 16 wt %, 17 wt %, 18 wt %, 19 wt % or 20 wt % based on the weight of the composition.

For white compositions, the amount of white pigment generally is present in an amount of from at or about 1 wt % to at or about 60 wt % based on the weight of the composition. In some applications, greater than 60 wt % white pigment can be present. Preferred white pigments include titanium dioxide (anatase and rutile), zinc oxide, lithopone (calcined coprecipitate of barium sulfate and zinc sulfide), zinc sulfide, blanc fixe and alumina hydrate and combinations thereof, although any of these can be combined with calcium carbonate. In some applications, a white ink can include 60 wt % or less white pigment, or 55 wt % or less white pigment, or 50 wt % white pigment, or 45 wt % white pigment, or 40 wt % white pigment, or 35 wt % white pigment, or 30 wt % white pigment, or 25 wt % white pigment, or 20 wt % white pigment, or 15 wt % white pigment, or 10 wt % white pigment, based on the weight of the composition. In some applications, a white ink can include 5 wt % to 60 wt %, or 5 wt % to 55 wt %, or 10 wt % to 50 wt %, or 10 wt % to 25 wt %, or 25 wt % to 50 wt %, or 5 wt % to 15 wt %, or 40 wt % to 60 wt % white pigment based on the weight of the composition. In some applications, a non-white ink can an amount of dye/pigment that is 5%, 6 wt %, 7 wt %, 8 wt %, 9 wt %, 10 wt %, 11 wt %, 12 wt %, 13 wt %, 14 wt %, 15%, 16 wt %, 17 wt %, 18 wt %, 19 wt %, 20 wt %, 21 wt %, 22 wt %, 23 wt %, 24 wt %, 25%, 26 wt %, 27 wt %, 28 wt %, 29 wt %, 30 wt %, 31 wt %, 32 wt %, 33 wt %, 34 wt %, 35%, 36 wt %, 37 wt %, 38 wt %, 39 wt %, 40 wt %, 41 wt %, 42 wt %, 43 wt %, 44 wt %, 45%, 46 wt %, 47 wt %, 48 wt %, 49 wt %, 50 wt %, 51 wt %, 52 wt %, 53 wt %, 54 wt %, 55%, 56 wt %, 57 wt %, 58 wt %, 59 wt % or 60 wt % based on the weight of the composition.

In some aspects, the additive or dopant comprises a conductive additive. Exemplary conductive additives include, but are not limited to graphite, graphite powder, carbon nanotubes, and metallic particles or nanoparticles, such as gold nanoparticles. In some aspects, the conductive additive is biocompatible and non-toxic.

In some aspects, the additive is a biologically active agent. The term “biologically active agent” as used herein refers to any molecule which exerts at least one biological effect in vivo. For example, the biologically active agent can be a therapeutic agent to treat or prevent a disease state or condition in a subject. Biologically active agents include, without limitation, organic molecules, inorganic materials, proteins, peptides, nucleic acids (e.g., genes, gene fragments, gene regulatory sequences, and antisense molecules), nucleoproteins, polysaccharides, glycoproteins, and lipoproteins. Classes of biologically active compounds that can be incorporated into the composition provided herein include, without limitation, anticancer agents, antibiotics, analgesics, anti-inflammatory agents, immunosuppressants, enzyme inhibitors, antihistamines, anti-convulsants, hormones, muscle relaxants, antispasmodics, ophthalmic agents, prostaglandins, anti-depressants, anti-psychotic substances, trophic factors, osteoinductive proteins, growth factors, and vaccines.

The term “active agent” may also be used herein to refer to a biological sample (e.g., a sample of tissue or fluid, such as for instance blood) or a component thereof, and/or to a biologically active entity or compound, and/or to a structurally or functionally labile entity.

Exemplary active agents include, but are not limited to, therapeutic agents, diagnostic agents (e.g., contrast agents), and any combinations thereof. In some embodiments, the active agent present in a silk matrix (e.g., a silk microsphere), composition, or the like can include a labile active agent, e.g., an agent that can undergo chemical, physical, or biological change, degradation and/or deactivation after exposure to a specified condition, e.g., high temperatures, high humidity, light exposure, and any combinations thereof. In some embodiments, the active agent present in the silk matrix (e.g., a silk microsphere), composition, or the like can include a temperature-sensitive active agent, e.g., an active agent that will lose at least about 30% or more, of its original activity or bioactivity, upon exposure to a temperature of at least about 10° C. or above, including at least about 15° C. or above, at least about room temperature or above, or at least about body temperature (e.g., about 37° C.) or above.

The active agent can be generally present in the silk matrix (e.g., a silk microsphere), composition, or the like in an amount of about 0.01% (w/w) to about 70% (w/w), or about 0.1% (w/w) to about 50% (w/w), or about 1% (w/w) to about 30% (w/w). The active agent can be present on a surface of the silk matrix (e.g., a silk microsphere), composition, or the like and/or encapsulated and dispersed in the silk matrix (e.g., a silk microsphere), composition, or the like homogeneously or heterogeneously or in a gradient. In some embodiments, the active agent can be added into the silk solution, which is then subjected to the methods described herein for preparing a silk matrix (e.g., a silk microsphere), composition, or the like. In some embodiments, the active agent can be coated on a surface of the silk matrix (e.g., a silk microsphere), composition, or the like. In some embodiments, the active agent can be loaded in a silk matrix (e.g., a silk microsphere), composition, or the like by incubating the silk microsphere in a solution of the active agent for a period of time, during which an amount of the active agent can diffuse into the silk matrix (e.g., a silk microsphere), composition, or the like, and thus distribute within the silk matrix (e.g., a silk microsphere), composition, or the like.

In some aspects, the additive is a therapeutic agent. As used herein, the term “therapeutic agent” means a molecule, group of molecules, complex or substance administered to an organism for diagnostic, therapeutic, preventative medical, or veterinary purposes. As used herein, the term “therapeutic agent” includes a “drug” or a “vaccine.” This term include externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term can also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a therapeutic effect, for example deoxyribonucleic acid (DNA), ribonucleic acid (RNA), nucleic acid analogues (e.g., locked nucleic acid (LNA), peptide nucleic acid (PNA), xeno nucleic acid (XNA)), or mixtures or combinations thereof, including, for example, DNA nanoplexes, siRNA, microRNA, shRNA, aptamers, ribozymes, decoy nucleic acids, antisense nucleic acids, RNA activators, and the like. Generally, any therapeutic agent can be included in the composition provided herein.

The term “therapeutic agent” also includes an agent that is capable of providing a local or systemic biological, physiological, or therapeutic effect in the biological system to which it is applied. For example, the therapeutic agent can act to control infection or inflammation, enhance cell growth and tissue regeneration, control tumor growth, act as an analgesic, promote anti-cell attachment, and enhance bone growth, among other functions. Other suitable therapeutic agents can include anti-viral agents, hormones, antibodies, or therapeutic proteins. Other therapeutic agents include prodrugs, which are agents that are not biologically active when administered but, upon administration to a subject are converted to biologically active agents through metabolism or some other mechanism. Additionally, a silk-based drug delivery composition can contain one therapeutic agent or combinations of two or more therapeutic agents.

A therapeutic agent can include a wide variety of different compounds, including chemical compounds and mixtures of chemical compounds, e.g., small organic or inorganic molecules; saccharines; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; an extract made from biological materials such as bacteria, plants, fungi, or animal cells; animal tissues; naturally occurring or synthetic compositions; and any combinations thereof. In some aspects, the therapeutic agent is a small molecule.

The term “bioactivity,” as used herein in reference to an active agent, generally refers to the ability of an active agent to interact with a biological target and/or to produce an effect on a biological target. For example, bioactivity can include, without limitation, elicitation of a stimulatory, inhibitory, regulatory, toxic or lethal response in a biological target. The biological target can be a molecule or a cell. For example, a bioactivity can refer to the ability of an active agent to modulate the effect/activity of an enzyme, block a receptor, stimulate a receptor, modulate the expression level of one or more genes, modulate cell proliferation, modulate cell division, modulate cell morphology, or any combination thereof. In some instances, a bioactivity can refer to the ability of a compound to produce a toxic effect in a cell. Exemplary cellular responses include, but are not limited to, lysis, apoptosis, growth inhibition, and growth promotion; production, secretion, and surface expression of a protein or other molecule of interest by the cell; membrane surface molecule activation including receptor activation; transmembrane ion transports; transcriptional regulations; changes in viability of the cell; changes in cell morphology; changes in presence or expression of an intracellular component of the cell; changes in gene expression or transcripts; changes in the activity of an enzyme produced within the cell; and changes in the presence or expression of a ligand and/or receptor (e.g., protein expression and/or binding activity). Methods for assaying different cellular responses are well known to one of skill in the art, e.g., western blot for determining changes in presence or expression of an endogenous protein of the cell, or microscopy for monitoring the cell morphology in response to the active agent, or FISH and/or qPCR for the detection and quantification of changes in nucleic acids. Bioactivity can be determined in some embodiments, for example, by assaying a cellular response.

In reference to an antibody, the term “bioactivity” includes, but is not limited to, epitope or antigen binding affinity, the in vivo and/or in vitro stability of the antibody, the immunogenic properties of the antibody, e.g., when administered to a human subject, and/or the ability to neutralize or antagonize the bioactivity of a target molecule in vivo or in vitro. The aforementioned properties or characteristics can be observed or measured using art-recognized techniques including, but not limited to, scintillation proximity assays, ELISA, ORIGEN immunoassay (IGEN), fluorescence quenching, fluorescence ELISA, competitive ELISA, SPR analysis including, but not limited to, SPR analysis using a BIAcore biosenser, in vitro and in vivo neutralization assays (see, for example, International Publication No. WO 2006/062685), receptor binding, and immunohistochemistry with tissue sections from different sources including human, primate, or any other source as needed. In reference to an immunogen, the “bioactivity” includes immunogenicity, the definition of which is discussed in detail later. In reference to a virus, the “bioactivity” includes infectivity, the definition of which is discussed in detail later. In reference to a contrast agent, e.g., a dye, the “bioactivity” refers to the ability of a contrast agent when administered to a subject to enhance the contrast of structures or fluids within the subject's body. The bioactivity of a contrast agent also includes, but is not limited to, its ability to interact with a biological environment and/or influence the response of another molecule under certain conditions.

As used herein, the term “small molecule” can refer to compounds that are “natural product-like,” however, the term “small molecule” is not limited to “natural product-like” compounds. Rather, a small molecule is typically characterized in that it contains several carbon-carbon bonds, and has a molecular weight of less than 5000 Daltons (5 kDa), preferably less than 3 kDa, still more preferably less than 2 kDa, and most preferably less than 1 kDa. In some cases it is preferred that a small molecule have a molecular weight equal to or less than 700 Daltons.

Exemplary therapeutic agents include, but are not limited to, those found in Harrison's Principles of Internal Medicine, 13th Edition, Eds. T. R. Harrison et al. McGraw-Hill N.Y., NY; Physicians' Desk Reference, 50th Edition, 1997, Oradell New Jersey, Medical Economics Co.; Pharmacological Basis of Therapeutics, 8th Edition, Goodman and Gilman, 1990; United States Pharmacopeia, The National Formulary, ETSP XII NF XVII, 1990, the complete contents of all of which are incorporated herein by reference.

Therapeutic agents include the herein disclosed categories and specific examples. It is not intended that the category be limited by the specific examples. Those of ordinary skill in the art will recognize also numerous other compounds that fall within the categories and that are useful according to the present disclosure. Examples include a radiosensitizer, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, an anticholinergic/antispasmodic agent, a vasopressin analogue, an anti arrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifungal agent, a vaccine, a protein, or a nucleic acid. In a further aspect, the pharmaceutically active agent can be coumarin, albumin, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritis antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetaminophen, ibuprofen, ketoprofen and piroxicam; analgesic agents such as salicylates; calcium channel blockers such as nifedipine, amlodipine, and nicardipine; angiotensin converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists such as clonidine; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; antiarrhythmic agents such as quinidine, lidocaine, tocainide hydrochloride, mexiletine hydrochloride, digoxin, verapamil hydrochloride, propafenone hydrochloride, flecainide acetate, procainamide hydrochloride, moricizine hydrochloride, and disopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as Coumadin, warfarin, acetylsalicylic acid, and ticlopidine; sedatives such as benzodiazepines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hdyrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides.

Anti-cancer agents include alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists/antagonists, endothelinA receptor antagonists, retinoic acid receptor agonists, immuno-modulators, hormonal and antihormonal agents, photodynamic agents, and tyrosine kinase inhibitors.

Antibiotics include aminoglycosides (e.g., gentamicin, tobramycin, netilmicin, streptomycin, amikacin, neomycin), bacitracin, corbapenems (e.g., imipenem/cislastatin), cephalosporins, colistin, methenamine, monobactams (e.g., aztreonam), penicillins (e.g., penicillin G, penicillinV, methicillin, natcillin, oxacillin, cloxacillin, dicloxacillin, ampicillin, amoxicillin, carbenicillin, ticarcillin, piperacillin, mezlocillin, azlocillin), polymyxin B, quinolones, and vancomycin; and bacteriostatic agents such as chloramphenicol, clindanyan, macrolides (e.g., erythromycin, azithromycin, clarithromycin), lincomyan, nitrofurantoin, sulfonamides, tetracyclines (e.g., tetracycline, doxycycline, minocycline, demeclocyline), and trimethoprim. Also included are metronidazole, fluoroquinolones, and ritampin.

Enzyme inhibitors are substances which inhibit an enzymatic reaction. Examples of enzyme inhibitors include edrophonium chloride, N-methylphysostigmine, neostigmine bromide, physostigmine sulfate, tacrine, tacrine, 1-hydroxy maleate, iodotubercidin, p-bromotetramiisole, 10-(alpha-diethylaminopropionyl)-phenothiazine hydrochloride, calmidazolium chloride, hemicholinium-3,3,5-dinitrocatechol, diacylglycerol kinase inhibitor I, diacylglycerol kinase inhibitor II, 3-phenylpropargylamine, N°-monomethyl-Larginine acetate, carbidopa, 3-hydroxybenzylhydrazine, hydralazine, cl orgyline, deprenyl, hydroxylamine, iproniazid phosphate, 6-MeO-tetrahydro-9H-pyrido-indole, nialamide, pargyline, quinacrine, semi carb azide, tranylcypromine, N,N-diethylaminoethyl-2,2-diphenylvalerate hydrochloride, 3-isobutyl-1-methylxanthne, papaverine, indomethacind, 2-cyclooctyl-2-hydroxy ethylamine hydrochloride, 2,3-dichloro-a-methylbenzylamine (DCMB), 8,9-dichloro-2,3,4,5-tetrahydro-1H-2-benzazepine hydrochloride, p-amino glutethimide, p-aminoglutethimide tartrate, 3-iodotyrosine, alpha-methyltyrosine, acetazolamide, dichlorphenamide, 6-hydroxy-2-benzothiazolesulfonamide, and allopurinol.

Antihistamines include pyrilamine, chlorpheniramine, and tetrahydrazoline, among others.

Anti-inflammatory agents include corticosteroids, nonsteroidal anti-inflammatory drugs (e.g., aspirin, phenylbutazone, indomethacin, sulindac, tolmetin, ibuprofen, piroxicam, and fenamates), acetaminophen, phenacetin, gold salts, chloroquine, D-Penicillamine, methotrexate colchicine, allopurinol, probenecid, and sulfinpyrazone.

Muscle relaxants include mephenesin, methocarbomal, cyclobenzaprine hydrochloride, trihexylphenidyl hydrochloride, levodopa/carbidopa, and biperiden.

Anti-spasmodics include atropine, scopolamine, oxyphenonium, and papaverine.

Analgesics include aspirin, phenybutazone, idomethacin, sulindac, tolmetic, ibuprofen, piroxicam, fenamates, acetaminophen, phenacetin, morphine sulfate, codeine sulfate, meperidine, nalorphine, opioids (e.g., codeine sulfate, fentanyl citrate, hydrocodone bitartrate, loperamide, morphine sulfate, noscapine, norcodeine, normorphine, thebaine, nor-binaltorphimine, buprenorphine, chlomaltrexamine, funaltrexamione, nalbuphine, nalorphine, naloxone, naloxonazine, naltrexone, and naltrindole), procaine, lidocain, tetracaine and dibucaine. [0121] Ophthalmic agents include sodium fluorescein, rose bengal, methacholine, adrenaline, cocaine, atropine, alpha-chymotrypsin, hyaluronidase, betaxalol, pilocarpine, timolol, timolol salts, and combinations thereof.

Prostaglandins are art recognized and are a class of naturally occurring chemically related long-chain hydroxy fatty acids that have a variety of biological effects.

Anti-depressants are substances capable of preventing or relieving depression.

Examples of anti-depressants include imipramine, amitriptyline, nortriptyline, protriptyline, desipramine, amoxapine, doxepin, maprotiline, tranylcypromine, phenelzine, and isocarboxazide.

Trophic factors are factors whose continued presence improves the viability or longevity of a cell trophic factors include, without limitation, platelet-derived growth factor (PDGP), neutrophil-activating protein, monocyte chemoattractant protein, macrophage-inflammatory protein, platelet factor, platelet basic protein, and melanoma growth stimulating activity; epidermal growth factor, transforming growth factor (alpha), fibroblast growth factor, platelet-derived endothelial cell growth factor, insulin-like growth factor, glial derived growth neurotrophic factor, ciliary neurotrophic factor, nerve growth factor, bone growth/cartilage-inducing factor (alpha and beta), bone morphogenetic proteins, interleukins (e.g., interleukin inhibitors or interleukin receptors, including interleukin 1 through interleukin 10), interferons (e.g., interferon alpha, beta and gamma), hematopoietic factors, including erythropoietin, granulocyte colony stimulating factor, macrophage colony stimulating factor and granulocyte-macrophage colony stimulating factor; tumor necrosis factors, and transforming growth factors (beta), including beta-1, beta-2, beta-3, inhibin, and activin.

Hormones include estrogens (e.g., estradiol, estrone, estriol, diethylstibestrol, quinestrol, chlorotrianisene, ethinyl estradiol, mestranol), anti-estrogens (e.g., clomiphene, tamoxifen), progestins (e.g., medroxyprogesterone, norethindrone, hydroxyprogesterone, norgestrel), antiprogestin (mifepristone), androgens (e.g, testosterone cypionate, fluoxymesterone, danazol, testolactone), anti-androgens (e.g., cyproterone acetate, flutamide), thyroid hormones (e.g., triiodothyronne, thyroxine, propylthiouracil, methimazole, and iodixode), and pituitary hormones (e.g., corticotropin, sumutotropin, oxytocin, and vasopressin). Hormones are commonly employed in hormone replacement therapy and/or for purposes of birth control. Steroid hormones, such as prednisone, are also used as immunosuppressants and anti-inflammatoires. In some aspects, the additive is an agent that stimulates tissue formation, and/or healing and regrowth of natural tissues, and any combinations thereof. Agents that increase formation of new tissues and/or stimulates healing or regrowth of native tissue at the site of injection can include, but are not limited to, fibroblast growth factor (FGF), transforming growth factor-beta (TGF-beta, platelet-derived growth factor (PDGF), epidermal growth factors (EGFs), connective tissue activated peptides (CTAPs), osteogenic factors including bone morphogenic proteins, heparin, angiotensin II (A-II) and fragments thereof, insulin-like growth factors, tumor necrosis factors, interleukins, colony stimulating factors, erythropoietin, nerve growth factors, interferons, biologically active analogs, fragments, and derivatives of such growth factors, and any combinations thereof.

In some aspects, the silk composition can further comprise at least one additional material for soft tissue augmentation, e.g., dermal filler materials, including, but not limited to, poly (methyl methacrylate) microspheres, hydroxylapatite, poly (L-lactic acid), collagen, elastin, and glycosaminoglycans, hyaluronic acid, commercial dermal filler products such as BOTOX® (from Allergan), DYSPORT®, COSMODERM®, EVOLENCER, RADIESSE®, RESTYLANE®, JUVEDERM® (from Allergan), SCULPTRA®, PERLANE®, and CAPTIQEIE®, and any combinations thereof.

In some aspects, the additive is a wound healing agent. As used herein, a “wound healing agent” is a compound or composition that actively promotes wound healing process.

Exemplary wound healing agents include, but are not limited to dexpanthenol; growth factors; enzymes, hormones; povidon-iodide; fatty acids; anti-inflammatory agents; antibiotics; antimicrobials; antiseptics; cytokines; thrombin; angalgesics; opioids; aminoxyls; furoxans; nitrosothiols; nitrates and anthocyanins; nucleosides, such as adenosine; and nucleotides, such as adenosine diphosphate (ADP) and adenosine triphosphate (ATP); neutotransmitter/neuromodulators, such as acetylcholine and 5-hydroxytryptamine (serotonin/5-HT); histamine and catecholamines, such as adrenalin and noradrenalin; lipid molecules, such as 5 sphingosine-1-phosphate and lysophosphatidic acid; amino acids, such as arginine and lysine; peptides such as the bradykinins, substance P and calcium gene-related peptide (CGRP); nitric oxide; and any combinations thereof.

In certain aspects, the active agents provided herein are immunogens. In one aspect, the immunogen is a vaccine. Most vaccines are sensitive to environmental conditions under which they are stored and/or transported. For example, freezing may increase reactogenicity (e.g., capability of causing an immunological reaction) and/or loss of potency for some vaccines (e.g., HepB, and DTaP/IPV/FQB), or cause hairline cracks in the container, leading to contamination. Further, some vaccines (e.g., BCG, Varicella, and MMR) are sensitive to heat. Many vaccines (e.g., BCG, MMR, Varicella, Meningococcal C Conjugate, and most DTaP-containing vaccines) are light sensitive. See, e.g., Galazka et ak, Thermostability of vaccines, in Global Programme for Vaccines & Immunization (World Health Organization, Geneva, 1998); Peetermans et ak, Stability of freeze-dried rubella virus vaccine (Cendehill strain) at various temperatures, 1 J. Biological Standardization 179 (1973). Thus, the compositions and methods provided herein also provide for stabilization of vaccines regardless of the cold chain and/or other environmental conditions.

In some aspects, the additive is a cell, e.g., a biological cell. Cells useful for incorporation into the composition can come from any source, e.g., mammalian, insect, plant, etc. In some aspects, the cell can be a human, rat or mouse cell. In general, cells to be used with the compositions provided herein can be any types of cells. In general, the cells should be viable when encapsulated within compositions. In some aspects, cells that can be used with the composition include, but are not limited to, mammalian cells (e.g. human cells, primate cells, mammalian cells, rodent cells, etc.), avian cells, fish cells, insect cells, plant cells, fungal cells, bacterial cells, and hybrid cells. In some aspects, exemplary cells that can be can be used with the compositions include platelets, activated platelets, stem cells, totipotent cells, pluripotent cells, and/or embryonic stem cells. In some aspects, exemplary cells that can be encapsulated within compositions include, but are not limited to, primary cells and/or cell lines from any tissue. For example, cardiomyocytes, myocytes, hepatocytes, keratinocytes, melanocytes, neurons, astrocytes, embryonic stem cells, adult stem cells, hematopoietic stem cells, hematopoietic cells (e.g. monocytes, neutrophils, macrophages, etc.), ameloblasts, fibroblasts, chondrocytes, osteoblasts, osteoclasts, neurons, sperm cells, egg cells, liver cells, epithelial cells from lung, epithelial cells from gut, epithelial cells from intestine, liver, epithelial cells from skin, etc, and/or hybrids thereof, can be included in the silk/platelet compositions disclosed herein. Those skilled in the art will recognize that the cells listed herein represent an exemplary, not comprehensive, list of cells. Cells can be obtained from donors (allogenic) or from recipients (autologous). Cells can be obtained, as a non-limiting example, by biopsy or other surgical means known to those skilled in the art.

In some aspects, the cell can be a genetically modified cell. A cell can be genetically modified to express and secrete a desired compound, e.g. a bioactive agent, a growth factor, differentiation factor, cytokines, and the like. Methods of genetically modifying cells for expressing and secreting compounds of interest are known in the art and easily adaptable by one of skill in the art.

Differentiated cells that have been reprogrammed into stem cells can also be used.

For example, human skin cells reprogrammed into embryonic stem cells by the transduction of Oct3/4, Sox2, c-Myc and Klf4 (Junying Yu, et. ah, Science, 2007, 318, 1917-1920 and Takahashi K. et. ah, Cell, 2007, 131, 1-12).

While the disclosure has been disclosed in connection with the preferred embodiments shown and described in detail, various modifications and improvements thereon will become readily apparent to those skilled in the art. Accordingly, the spirit and scope of the present disclosure is not to be limited by the foregoing examples, but is to be understood in the broadest sense allowable by law.

### EXAMPLES

### Example 1

### Materials/Methods:

Silk Isolation—Silk fibroin was isolated from Bombyx mori cocoons as previously described. (See Xiao L, Lu G, Lu Q, Kaplan D L. Direct Formation of Silk Nanoparticles for Drug Delivery. ACS Biomater Sci Eng. 2016; 2(11):2050-7) Briefly, the cocoons (Tajima Shoji Co., Japan) were boiled in a 0.02 M sodium carbonate solution for 10, 30, or 60 min to remove the sericin and achieve different molecular weights of silk, which we have previously published (See Pritchard E M, Hu X, Finley V, Kuo C K, Kaplan D L. Effect of silk protein processing on drug delivery from silk films. Macromol Biosci. 2013; 13(3):311-20.): The extracted silk was then dried for 12 h in a chemical hood before being dissolved in a 9.3 M lithium bromide solution at 60° C. for 4 h, yielding a 20% w/v solution. This solution was dialyzed against deionized (DI) water using Pierce Slide-a-Lyzer cassettes, MWCO 3,500 Da (Rockford, IL) for 3 days to remove the lithium bromide. The solution was centrifuged (9,000 rpm, 4° C., 20 min cycle), and a final concentration of the aqueous silk fibroin of ˜5-10% w/v was obtained. For the 10 min extracted silk, extra rinse steps (3-5) immediately after boiling were employed to effectively remove any residual sericin, as any residual sericin prohibits particle formation.

Fabrication of SNPs—Nanoprecipitated SNPs were prepared as previously published but with modifications. (See Wongpinyochit T, Johnston B F, Seib F P. Manufacture and Drug Delivery Applications of Silk Nanoparticles. J Vis Exp. 2016(116) and Xiao L, Lu G, Lu Q, Kaplan D L. Direct Formation of Silk Nanoparticles for Drug Delivery. ACS Biomater Sci Eng. 2016; 2(11):2050-7.) Briefly, a 5-10% w/v silk solution was added dropwise using a 60 mL addition funnel (Fisher Scientific CG170401, Waltham, MA) at a drop length of 7 cm and drop rate of approximately 8-10 drop/min to acetone while stirring (200 rpm-1,200 rpm, Cole Palmer UX-84003-80, Vernon Hills, IL) with a 15×4 mm stir bar to create a cloudy solution that was >75% v/v acetone (typically, 3.5-4 mL of silk solution was added to ˜17.5-18 mL acetone in a 20 mL scintillation vial). Cold bath particles were prepared by adding 6% w/v silk solution dropwise to −20° C. acetone while stirring at 800 rpm. The nanoparticle suspension was allowed to stir for 48-72 hr to evaporate all the solvent. The nanoparticle suspension was then diluted with deionized water, and sonicated with a Branson Ultrasonic Cell Disruptor for 30 seconds at 30% amplitude. For each experiment, nanoparticles were prepared in replicates (typically in at least triplicate unless otherwise stated), where different batches of silk were dissolved to generate each nanoparticle suspension replicate. To make fluorescently labeled SNPs, fluorescein isothiocyanate (FITC) was dissolved in dimethyl sulfoxide (DMSO) to achieve a 10 mg/mL solution. Per 100 mg of silk solution needed for the nanoprecipitation process, 1 mg of FITC was added (e.g., for 4 mL of a 5% w/v silk solution, 200 μL of the FITC stock solution was added to add 2 mg of FITC to 200 mg of silk). After adding the FITC to the silk solution and ensuring homogeneous mixing, the nanoprecipitation process was conducted. Any unbound FITC was dialyzed and/or ultracentrifuged at 60,000 rpm, 4° C., for 30 min (Beckman Coulter Optima Max TL with TLA-110 rotor, Brea, CA) from the SNPs with several wash cycles until the supernatants revealed no leached or unbound FITC using a plate reader (491 nm excitation and 516 nm emission, Varioskan ThermoFisher, Waltham, MA).

Size quantification—The size, PDI, and zeta potential of the SNPs were measured using a ZetaPALS Dynamic Light Scattering (DLS) machine (Brookhaven Instruments, Holtzville, NY). Of note, size and PDI measurements were taken three times on the DLS machine for each sample (technical replicates), and then averaged for each batch of SNPs dissolved from a fresh batch of silk (biological replicates). Because of this, the variability of sizes may be higher than the variability of the PDI between different biological replicates. To verify the readings, scanning electron microscopy (SEM) and transmission electron microscopy (TEM) were utilized. For SEM, the nanoparticles were coated with a thin layer (10 nm thick) of Pt/Pd using a sputter coater (208 HR, Cressington Scientific Instruments Inc.) and imaged (Zeiss UltraPlus SEM or Zeiss Supra 55 VP SEM, Carl Zeiss SMT Inc.) at a voltage of 3 kV.

For TEM, 2% ammonium molybdate stain was prepared and the pH adjusted to 6.9 using sodium hydroxide. The stain was filtered through a 0.2 μm filter just before use. Carbon-coated Cu grids (Ted Pella, Inc., 400 mesh size, Redding, CA) were glow discharged for 30 seconds using a PELCO easiGlow. All grids were prepared within 30 min of glow discharge. 5 μL of sample was pipetted directly onto the grid for 30 seconds before blotting using filter paper (Whatman 1. Thermofisher, 09805F, Waltham, MA). The grid was immediately placed on a 50 μL drop of 2% ammonium molybdate, pH 6.9, for 15 seconds before blotting. This staining step was repeated twice, blotting between each step. After the final blot, the filter paper was pressed to the edge of the grid near the tweezers to remove excess stain. Grids were dried sample side down on filter paper overnight. Images were collected on the Tecnai T12 in the San Diego State University Electron Microscope facility at 120 kV and spot size 3 using a side-mounted CCD camera.

Cell culture, uptake, and staining—U87-MG (ATCC® HTB-14 Manassas, VA) glioblastoma cells were cultured in Dulbecco's Modified Eagle's Medium (DMEM)-F12 supplemented with 1% antibiotic-antimycotic (Sigma-Aldrich) and 10% fetal bovine serum (FBS). For live cell imaging, 100,000 cells/well were seeded in 6 well plates and left to recover for two days. Media containing FITC-labeled particles was added at 16.5 μg/mL for four hours. The media was then aspirated and media containing Lysotracker Deep Red (ThermoFisher, L12492, Waltham, MA) was incubated with the cells for 30-40 min according to the manufacturer's instructions. Lysotracker media was aspirated, and fresh media was added to the wells. Trypan blue dye was added to the wells in a 1:2 ratio of Trypan blue to media to quench fluorescence from extracellular particles and visualize only intracellular FITC-tagged particles. Cells were imaged on a Keyence All-in-One Fluorescent Microscope (BZ-X710, Keyence Corp, Osaka, Japan) within 30 min of adding Trypan Blue. All images shown were taken with the same exposure levels.

For fixed cell imaging, similar methods were employed. Here, 100,000 cells/well were seeded in 6 well plates and left to recover for two days. Media containing FITC-labeled particles was added at 16.5 μg/mL for four hours. U87-MG cells were fixed with 4% paraformaldehyde for 30 min, permeabilized with 0.1% Triton X-100 at room temperature (RT) in 1% blocking buffer (Abcam, ab210904, Waltham, MA) for 10 min and blocked with 1% blocking buffer for 45 min to 1 h. The permeabilized cells were incubated with an antibody for lysosome-associated membrane glycoprotein 2 (LAMP2, Thermofisher PA1655, Waltham, MA) overnight at 4° C. while shaking, and then the early endosome antibody EEA1 (Thermofisher MA514794, Waltham, MA). To visualize if the particles were in locations other than the endosomes and lysosomes, the same secondary antibody (labeled with AlexaFluor 405, Thermofisher, A31556, Waltham, MA) was used for both, as both primary antibodies utilized rabbit hosts. Incubation of the secondary antibody was conducted in the dark with shaking at RT. Finally, phalloidin was added according to the manufacturer's instructions for 30 min at RT in the dark with shaking (labeled with AlexaFluor 555, Thermofisher, A34055, Waltham, MA). All dilutions of the antibodies were done according to the manufacturer's instructions (LAMP2—1:20, EEA1—1:100, secondary antibody labeled with AlexaFluor 405—1:200, Phalloidin—1:200). Between the sequential staining steps, samples were washed in PBS containing 0.1% Tween-20. Images were taken within 24 h of staining using a Leica SP2 confocal microscope (Leica Microsystems) and Nikon A1R (Nikon Instruments Inc.) with Z-series capability. Images were assembled with Leica confocal software (ver 2.61, Leica), NIS-Elements AR software package (ver 4.20.01, Nikon) and ImageJ.

Drug loading and in vitro release profile—To entrap DOX in the SNPs, the drug was added directly to silk solution prior to nanoprecipitation (ESNPs). Briefly, a 10 mg/mL solution of DOX was generated in DMSO, and 200 μL of this solution (2 mg) was added to 4 mL of silk solution used for nanoprecipitation. Immediately following nanoprecipitation of the ESNPs, the silk-acetone-doxorubicin mixture (21-22 mL) was ultracentrifuged (3×, 60,000 RPM, 30 min, 4° C.) to remove unbound DOX and acetone. Supernatants were saved each time for quantification of loading. To coat the SNPs with DOX (CSNPs), similar protocols were used with modifications (See Wongpinyochit T, Johnston B F, Seib F P. Manufacture and Drug Delivery Applications of Silk Nanoparticles. J Vis Exp. 2016(116).) to compare the ESNPs to the CSNPs. Using one batch of premade, unloaded SNPs, 200 μL (2 mg) of DOX stock solution (DMSO) was added, the volume was brought to 21-22 mL using distilled water, and left to incubate overnight at RT while stirring. After the overnight incubation for CSNPs, the particles were ultracentrifuged 3×, 60,000 RPM, 30 min, 4° C.) to remove unbound DOX, and the supernatants were again saved for quantification of loading.

For the in vitro release profile, 500 μL of either ESNPs or CSNPs suspended in phosphate buffered saline (PBS) were pipetted into Slide-A-Lyzer 3.5K mini dialysis devices (ThermoFisher, PI69550, Waltham, MA), which were suspended in 1.8 mL of phosphate buffer pH 7.4, at 37° C. The 1.8 mL was saved for DOX quantification at each time point and replaced with fresh PBS. These experiments were done with 3 technical replicates per experiment, with 3 separate experiments (new batch of the ESNPs or CNSPs derived from freshly degummed silk), using two conditions: the 200 rpm, 6% w/v silk, 30 min extraction (mid MW) formulation, and the 1200 rpm, 5% w/v silk, 30 min extraction (mid MW) formulation.

The amount of DOX in the release samples was quantified with high-performance liquid chromatography (HPLC, Agilent Technologies) using previously established methods. (See Daeihamed M, Haeri A, Dadashzadeh S. A Simple and Sensitive HPLC Method for Fluorescence Quantitation of Doxorubicin in Micro-volume Plasma: Applications to Pharmacokinetic Studies in Rats. Iran J Pharm Res. 2015; 14(Suppl):33-42.) Briefly, an Agilent ZORBAX Eclipse Plus-C18 column (5 μm, 3×150 mm²) was used with a mobile phase of acetonitrile and water (32:68, v/v) that was pH adjusted to 2.6 using 85% orthophosphoric acid. The mobile phase was administered isocratically at a flow rate of 1 mL/min at 35° C. Samples were read at 475 nm excitation and 555 nm emission wavelengths, with an injection volume of 50 μL.

Positively charged particles—To generate cationic SNPs, silk was carbodiimide coupled with ethylenediamine (EDA) using 1-Ethyl-3-(3-dimethylaminopropyl) carbodiimide/N-hydroxy succinimide (EDC/NHS) bioconjugation chemistry. EDC and NHS were weighed and 0.2 M of 2-(N-morpholino)ethanesulfonic acid (MES) buffer (pH 6) was added to reach a final reaction concentration of 0.05 M as previously described. (See Hasturk O, Sahoo J K, Kaplan D L. Synthesis and Characterization of Silk Ionomers for Layer-by-Layer Electrostatic Deposition on Individual Mammalian Cells. Biomacromolecules. 2020; 21(7):2829-43.) Silk, MES buffer, EDC, NHS, and EDA were combined and stirred at 200 rpm for 18 h at RT. The silk solution was dialyzed for 72 h to remove the unbound EDA and other by-products, followed by centrifuging (9000 rpm, 20 min, 4° C.) to remove silk debris as previously described. (See Hasturk O, Sahoo J K, Kaplan D L. Synthesis and Characterization of Silk Ionomers for Layer-by-Layer Electrostatic Deposition on Individual Mammalian Cells. Biomacromolecules. 2020; 21(7):2829-43.) The nanoprecipitation protocol was then carried out using the cationic silk directly after chemical modification by concentrating to appropriate concentration or using lyophilized cationic silk that was reconstituted in deionized (DI) water to yield positively charged SNPs.

Antibody Conjugation—Antibody conjugation was conducted similarly to previously reported methods of attaching antibodies to silk films. (See Lu Q, Wang X, Zhu H, Kaplan D L. Surface immobilization of antibody on silk fibroin through conformational transition. Acta Biomater. 2011; 7(7):2782-6.) EDC and NHS were weighed and 0.05 M MES buffer (pH 6) was added similarly to previously described protocols. (See Hasturk O, Sahoo J K, Kaplan D L. Synthesis and Characterization of Silk Ionomers for Layer-by-Layer Electrostatic Deposition on Individual Mammalian Cells. Biomacromolecules. 2020; 21(7):2829-43.) Briefly, 12.4 mg of EDC and 4 mg of NHS were dissolved in 0.05 M MES buffer (pH 6) and brought to 2.4 mL with 5 mg of SNPs (200 rpm, 6% silk, and mid MW formulation). Then 10 μg of primary antibody (anti-epidermal growth factor receptor (EGFR), MA5-13319, Thermofisher Waltham, MA) was added to the MES, EDC/NHS, and SNPs mixture and stirred at 200 rpm for 18 h. The next day, this mixture was ultracentrifuged at 60,000 rpm for 30 min at 4° C. to pellet the particles. The pellet was resuspended in DI water and spun down 2 more times to wash. The particles were then resuspended in 3 mL of DI water, containing 0.2% blocking buffer (10×, Abcam, ab210904) and 0.01 mg/mL secondary antibody (AlexaFluor 405, A-31553, Thermofisher, Waltham, MA for 2 h shaking at RT. Blank SNPs were also incubated in 0.2% blocking buffer and 0.01 mg/mL secondary antibody as a control for nonspecific binding of the secondary antibody to the SNPs. The fluorescence of the particle aggregates was read using the Varioskan LUX Multimode Microplate Reader (401 nm excitation, 426 emission) and the particles were imaged on Keyence All-in-One Fluorescent Microscope. ESNPs (200 rpm, 6% silk, and mid MW formulation) were also tested for antibody conjugation of anti-EGFR for determination of successful antibody binding after drug loading. In order to see potential loss of doxorubicin/changes in doxorubicin fluorescence from the particles after antibody conjugation processes, the fluorescence of the particle aggregates was read using the Varioskan LUX Multimode Microplate Reader (470 nm excitation, 560 nm emission) before and after conjugation.

Statistical Analysis and Principal Component Analysis—All data are expressed as mean±standard deviation. GraphPad Prism (GraphPad Software, La Jolla, CA) was used to perform One-Way Analysis of Variance (ANOVA) with Tukey's multiple comparison post hoc test for most purposes unless otherwise stated. Principal component analysis (PCA) was utilized to determine the variables that contributed most to altering nanoparticle size. The data was standardized, and a covariance matrix was computed. The eigenvalues and eigenvectors of that matrix were computed to identify the principal components of the dataset. The percentage of variance in the data that can be explained by a single principal component was calculated. For each experiment unless otherwise stated, nanoparticles were evaluated in at least triplicate, where different batches of silk were dissolved to generate the nanoparticles for each “biological” replicate. Meaning, each “n” was evaluated using a freshly made batch of SNPs. Each biological replicate also had technical replicates.

Silk Hydrogel Fabrication We fabricated silk hydrogels embedded with nanoparticles by combining regenerated silk solution, horse-radish peroxidase, hydrogen peroxide, and distilled water. SNPs were included into the gel solution in the water for a final concentration of 4-5 mg/mL. Gel solution was transferred to a well plate and incubated at 37° C. for 1 hour to induce gelation. PBS was added to the surface of fully formed gels to prevent drying out prior to dynamic mechanical analysis (TA Instruments RSA3 Dynamic Mechanical Analyzer (TA Instruments, New Castle, DE)), which could alter measured properties. (See Hasturk O, Jordan K E, Choi J, Kaplan D L. Enzymatically crosslinked silk and silk-gelatin hydrogels with tunable gelation kinetics, mechanical properties and bioactivity for cell culture and encapsulation. Biomaterials. 2020; 232:119720).

1. A method comprising:
a) adding a silk solution dropwise into a volatile solvent that is miscible with water, thereby forming a precipitate-bearing solution, wherein the silk solution contains silk fibroin in an amount by weight of at least 2% and at most 25%, wherein the precipitate-bearing solution includes the volatile solvent in an amount of at least 75% (v/v);
b) applying shear forces to the precipitate-bearing solution, the applying continuing for a length of time sufficient to achieve evaporation of at least 95% of the volatile solvent, thereby producing a population of silk fibroin nanoparticles in water having a polydispersity index (PDI) of 0.35 or less.

2. The method of claim 1, wherein the applying of step b) is performed by stirring the precipitate-bearing solution.

3. The method of claim 2, wherein the stirring is performed with a magnetic stir bar.

4. The method of claim 2, wherein the stirring is performed at a temperature of between a freezing point of the precipitate-bearing solution and 60° C.

5. The method of claim 1, wherein the silk solution comprises an active agent, wherein the active agent is optionally doxorubicin, wherein a presence of the active agent in the silk solution results in the active agent being embedded within the population of silk fibroin nanoparticles.

6. The method of claim 1, wherein the volatile solvent is acetone, ether, an alcohol or other solvents having comparable miscibility and/or boiling points.

7. The method of claim 1, the method further comprising sonicating the population of silk fibroin nanoparticles.

8. The method of claim 1, the method further comprising crosslinking individual silk fibroin molecules within individual silk fibroin nanoparticles.

9. The method of claim 8, wherein the crosslinking is achieved by adding an enzymatic crosslinker to the population of silk fibroin nanoparticles, wherein the enzymatic crosslinker is optionally glutaraldehyde, transglutaminase, or peroxidase.

10. The method of claim 1, the method further comprising surface modifying the population of silk fibroin nanoparticles.

11. The method of claim 10, the surface modifying comprising affixing antibodies to the population of silk fibroin nanoparticles.

12. The method of claim 1, the method further comprising adjusting surface charge of the population of silk fibroin nanoparticles.

13. A composition made by the method of claim 1.

14. The composition of claim 13, wherein the composition is a hydrogel having the population of silk fibroin nanoparticles embedded therein.

15. The composition of claim 14, wherein the hydrogel is a silk fibroin hydrogel.

16. A method of administering silk fibroin nanoparticles comprising administering a first plurality of silk fibroin nanoparticles having an average diameter below a predetermined size threshold and a polydispersity index of 0.35 or less, wherein the first plurality of silk fibroin nanoparticles is capable of exiting lysosomes.

17. The method of claim 16, wherein the predetermined size threshold is <130 nm.

18. A method of tuning silk nanoparticle size distribution, the method comprising:
selecting, for inclusion in a silk solution, a predetermined molecular weight of silk fibroin and a predetermined concentration to produce a desired silk fibroin nanoparticle size distribution;
optionally selecting, for inclusion in the method, at least one operational parameter including a shear force, a stir speed, a stir bar size, a temperature, a drop length, or a drop rate to produce the desired silk fibroin nanoparticle size distribution;
adding the silk solution dropwise into a volatile solvent that is miscible with water, thereby forming a precipitate-bearing solution, wherein the precipitate-bearing solution includes the volatile solvent in an amount of at least 75% (v/v);
applying a shear force to the precipitate-bearing solution, the applying continuing for a length of time sufficient to achieve evaporation of at least 95% of the volatile solvent, thereby producing a population of silk fibroin nanoparticles in water having the desired silk fibroin nanoparticle size distribution,
wherein the desired silk fibroin nanoparticle size distribution includes a polydispersity index (PDI) of 0.35 or less.

19. The method of claim 18, wherein the silk solution contains silk fibroin in an amount by weight of at least 2% and at most 25%.

20. The method of claim 18, the method comprising the selecting, for inclusion in the method, of the at least one operational parameter including the shear force, the stir speed, the stir bar size, the temperature, the drop length, or the drop rate to produce the desired silk fibroin nanoparticle size distribution.

21.-37. (canceled)
